(19) United States
(12) Patent Application Publication
(10) Pub. No.: US 2015/0352114 A1
(43) Pub. Date: Dec. 10, 2015

(54) MITOTIC KINESIN Eg5 INHIBITING ANTICANCER AGENTS

(71) Applicant: THE CLEVELAND CLINIC FOUNDATION, Cleveland, OH (US)

(72) Inventors: Steven S. Rosenfeld, Cleveland, OH (US); Jeremy Rich, Cleveland, OH (US); Monica Venere, Cleveland, OH (US)

(21) Appl. No.: 14/678,310

(22) Filed: Apr. 3, 2015

Related U.S. Application Data
(60) Provisional application No. 61/974,850, filed on Apr. 3, 2014.

(51) Int. Cl. A61K 31/517; A61K 31/352; A61K 31/4741; A61K 31/5377; A61K 31/519; A61K 31/505; A61K 31/4709

(52) U.S. Cl. CPC A61K 31/517; A61K 31/505; A61K 31/352; A61K 31/4709; A61K 31/5377; A61K 31/519; A61K 31/4741

(57) ABSTRACT

A method of treating glioblastoma multiforme (GBM) in a subject in need thereof including administering to the subject a therapeutically effective amount of an Eg5 inhibitor.

### CROSS-REFERENCE TO RELATED APPLICATIONS

This application claims priority from U.S. Provisional Application Ser. No. 61/974,850, filed Apr. 3, 2014, the entire contents of which are incorporated herein by reference.

### GOVERNMENT FUNDING

This work was supported, at least in part, by award number CA172986 and GM076177 from the Department of Health and Human Services, National Institutes of Health. The United States government has certain rights in this invention.

### BACKGROUND

The prognosis for patients afflicted with glioblastoma (GBM) has remained grim, despite decades of translational and clinical investigation. Several features in particular contribute to the malignant phenotype of this disease. GBM is a highly proliferative tumor, with a mitotic fraction that ranges from 10-50%. This proliferative capacity is in part supported by a highly pro-angiogenic microenvironment. In addition, although GBM rarely metastasizes outside the central nervous system (CNS), it is capable of widely disseminating within the brain, a feature that severely limits the efficacy of surgery and radiotherapy. Each of these features appears to be driven in part by a subset of GBM cells that have stem cell-like properties and are referred to as glioblastoma stem cells (GSCs). GSCs are functionally defined by their self-renewal capacity and their ability to recapitulate the original patient-derived tumor in xenograft models. GSCs are resistant to radiotherapy and alkylating chemotherapy, drive angiogenesis, and are highly invasive, which contributes appreciably to the high rate of recurrence that is characteristic of GBM.

Mitosis, cell motility, and cell morphology all require the microtubule-based cytoskeleton, and these cellular physiologies are important not only for GBMs, but for a number of other highly aggressive malignancies as well. Several classes of microtubule poisons, including the taxanes, vinca alkaloids, and epothilones, have been used successfully in treating hematologic and solid malignancies. However, the microtubule-based cyto skeleton is crucial for CNS functions, including axonal transport, and neurotoxicity is the dose-limiting side effect of many of these microtubule poisons. This has led to efforts to identify and target microtubule-associated proteins (MAPs) whose inhibition would block mitosis without producing the neurotoxicity seen with microtubule poisons. One class of MAPs that appear to satisfy these requirements are a group of microtubule-based molecular motors, the mitotic kinesins, that orchestrate a number of steps in the mitotic process, including chromosome congression, formation of the mitotic spindle, kinetochore microtubule dynamics, and cytokinesis. Highly specific small molecule inhibitors directed against several of these mitotic kinesins have been developed in both preclinical models and in phase I and II clinical trials, and as expected, these drugs have not produced the neurotoxicity seen with microtubule poisons.

Eg5 (also known as Kif11) is a plus end directed, processive kinesin that is required for formation of the bipolar spindle in metaphase, where it opposes the action of minus end directed molecular motors. It is also the mitotic kinesin that has received the most attention in clinical trials and it is the target for over twenty high affinity, specific small molecule inhibitors that have been developed over the last 15 years and that all bind to the same structural motif in the catalytic domain of this molecular motor. Suppression of Eg5 function pharmacologically or by RNA interference leads to collapse of the mitotic spindle, mitotic catastrophe, and cell death. However, Eg5 also appears to have non-mitotic functions as well. It affects the dynamics of microtubule growth at the plus end in several forms of yeast, and may be capable of inducing microtubule polymerization at the microtubule plus end in metazoans. It also has been shown to regulate axonal branching and growth cone motility, through a mechanism that may involve sliding of parallel oriented microtubules in these structures. Finally, Eg5 has also been shown to be involved in cell motility.

### SUMMARY OF THE INVENTION

This application relates to methods for using Eg5 inhibitors, and pharmaceutical compositions comprising the same, to treat glioblastoma multiforme (GBM) in subjects in need thereof. One aspect of the application relates to a method of treating GBM in a subject in need thereof. The method includes administering to the subject a therapeutically effective amount of an Eg5 inhibitor. In some aspects, the Eg5 inhibitor may inhibit GBM metastasis. The glioblastoma in the subject may be characterized by the presence of glioma cancer stem cells. In some aspects, the GBM can include GBM of the brain. The subject in need of GBM treatment can be a human subject. In some aspects, the method can further include surgical resection of a GBM tumor in the subject.

In some aspects, the Eg5 inhibitor can include a small molecule. The small molecule can target a motif in the catalytic domain of Eg5. The Eg5 inhibitor can be selected from the group consisting of:

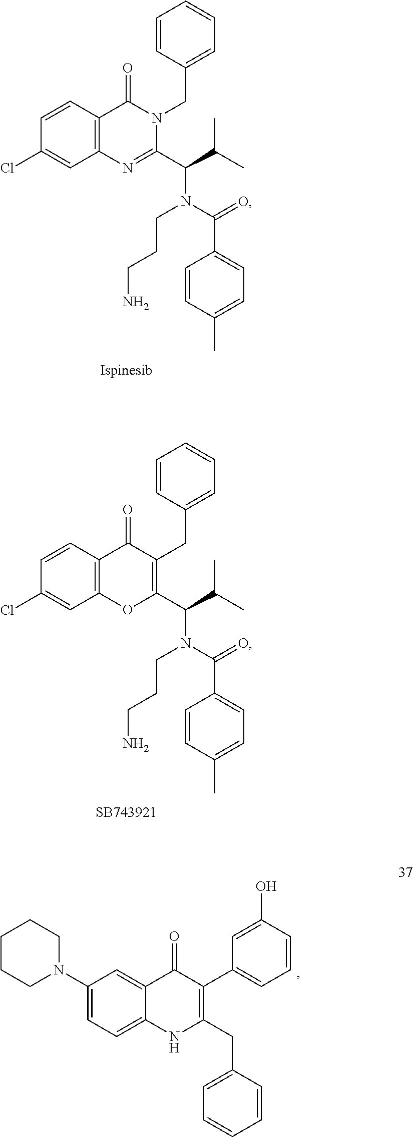 [SMILES: Cc1ccc(C(=O)N(CCCN)[C@@H](c2nc3cc(Cl)ccc3c(=O)n2Cc2ccccc2)C(C)C)cc1 ; Cc1ccc(C(=O)N(CCCN)[C@@H](c2oc3cc(Cl)ccc3c(=O)c2Cc2ccccc2)C(C)C)cc1 ; O=c1c(-c2cccc(O)c2)c(Cc2ccccc2)[nH]c2ccc(N3CCCCC3)cc12]

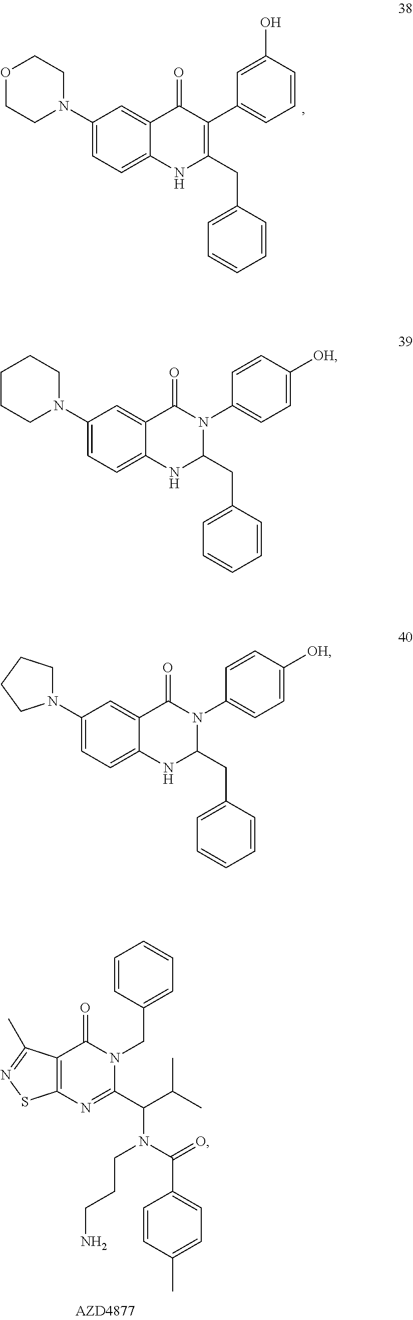 [SMILES: O=c1c(-c2cccc(O)c2)c(Cc2ccccc2)[nH]c2ccc(N3CCOCC3)cc12 ; O=C1c2cc(N3CCCCC3)ccc2NC(Cc2ccccc2)N1c1ccc(O)cc1 ; O=C1c2cc(N3CCCC3)ccc2NC(Cc2ccccc2)N1c1ccc(O)cc1 ; Cc1ccc(C(=O)N(CCCN)C(c2nc3snc(C)c3c(=O)n2Cc2ccccc2)C(C)C)cc1]

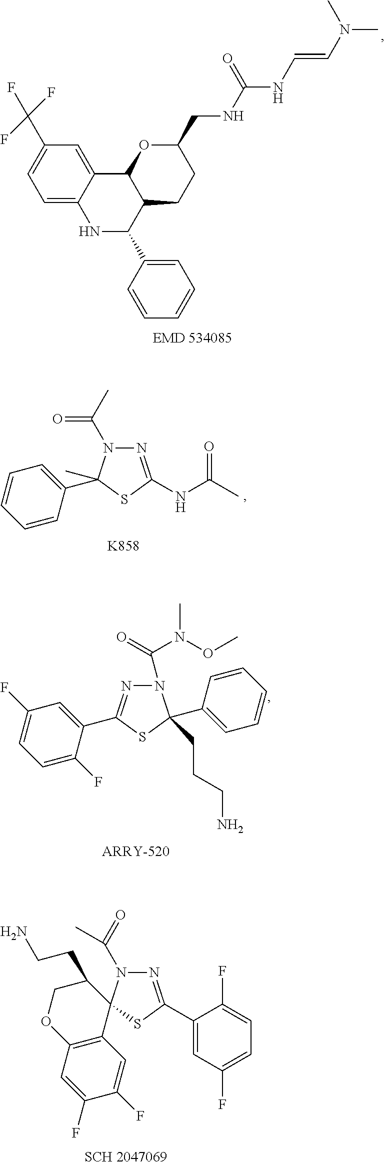

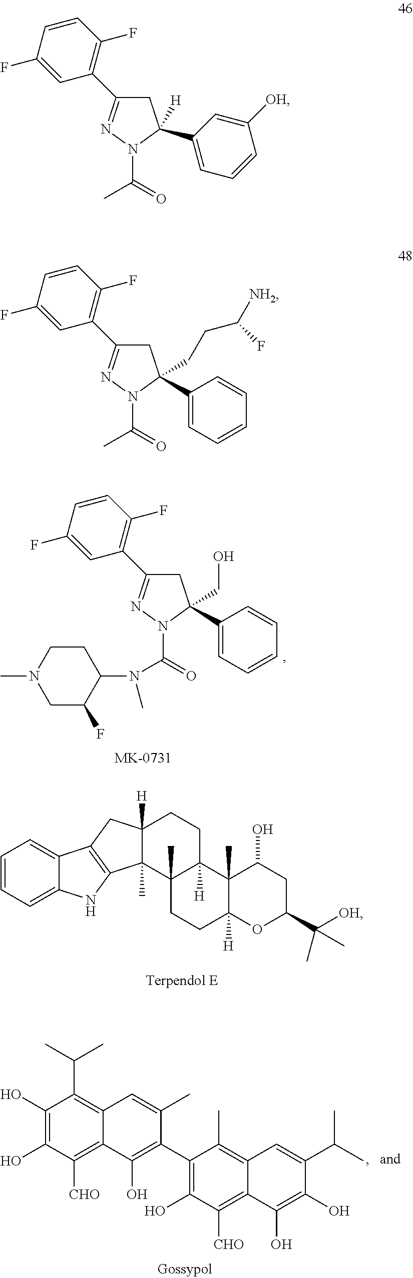

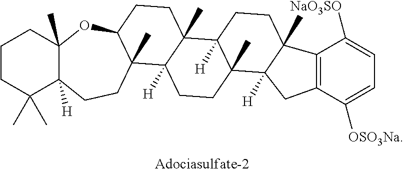

In some aspects, the Eg5 inhibitor can include ispinesib. In some aspects, the Eg5 inhibitor can be administered to the subject systemically.

### BRIEF DESCRIPTION OF THE FIGURES

The foregoing and other features of the application will become apparent to those skilled in the art to which the application relates upon reading the following description with reference to the accompanying drawings, in which:

FIG. 1A and FIGS. 1(B-E) illustrate that mitotic kinesins, including Eg5, are upregulated in GBM; (A) qPCR was performed for the listed mitotic regulators on RNA isolated from glioblastoma stem cells (GSCs) and matched NSTCs from three patient derived xenografts. Results are reported as fold change over matched NSTCs; (B) Eg5 protein was scored on a tissue micro-array containing normal brain control (n=9) and patient specimens from WHO grades II (n=25), III (n=22) and IV (n=47). p=0.0001, one-way ANOVA with post-hoc Tukey's test; (C) Patient survival data for high-grade glioma correlating with low or high-Eg5 expression was plotted in a Kaplan-Meier survival curve. p=0.047, log-rank test; (D) Representative H & E and immunohistochemistry (IHC) for Eg5 between normal brain and a GBM specimen; (E) Representative signal for Eg5 along the mitotic spindle of GBM cells. For all images, scale bars represent 10 μm.

FIG. 2(A-D) illustrate that Eg5 is elevated in GSCs due to attenuated protein turnover; (A) Whole cell lysates from matched GSCs and NSTCs from three patient derived xenografts were probed for Eg5, Olig2 and GFAP; (B) GSCs and NSTCs from xenograft specimen 3691 were synchronized at G1/S using a double-tymidine block. Following release, whole cell lysates were made every 2 hours over a 10-hour time course. Asynchronous (A) lysates were also harvested. Resulting lysates were probed for Eg5; (C) GCSs and NSTCs from xenograft specimen 08-387 were treated as in (B) with lysates harvested every 2 hours over a 24 hour time course; (D) GSCs and NSTCs from xenograft specimen 08-387 were synchronized at M using a nocodazole block. Lysates were made 4 hours following release at late-M early G1. APC^{Cdh1} activity was evaluated using HA-Securin as a substrate. Samples for immunoblot analysis were taken every 30 minutes over a 90 minute time course and probed for HA. For all immunoblots, βactin served as a loading control. Molecular weight (MW) of resulting bands is given in kilodaltons (kD).

FIGS. 3(A-C) illustrate that the viability of GSCs and NSTCs is targeted by Eg5 inhibition; (A) Matched GSCs and NSTCs from 6 patient derived xenografts were exposed to increasing concentrations of isinesib (0 to 32 nM) followed by analysis of cell viability using an ATP based assay. GSCs, EC₅₀=1.15±0.35 nM; NSTCs, EC₅₀=1.79±0.22 nM. Error bars represent s.d. p=0.0085; (B) Matched GSCs and NSTCs from 3 patient derived xenografts were evaluated for the percent of subG1 (apoptotic) cells over a 4 day time course following a single exposure to vehicle (DMSO) or 3 nM ispinesib at Day 0; (C) Matched GSCs and NSTCs from 2 patient derived xenografts were monitored for overall cell viability via an ATP based assay over a 5 day time course following a single exposure to vehicle (DMSO) or ispinesib. Error bars represent s.d.; **, p<0.01; ****, p<0.0001.

FIGS. 4(A-C) illustrate Eg5 inhibition targets GSCs in vivo; (A) Mice were subcutaneously injected in the flank with 100,000 bulk 3691 GBM cells. When tumors reached approximately 0.12 cm³, mice were randomized into one of two treatment groups; vehicle only (DMSO; n=5) or 10 mg/kg ispinesib (n=5) with daily administration over a 7 day time course. Arrows indicate day of vehicle or drug administration. Tumor volume was measured daily. Error bars represent s.d.; **, p<0.01; ***, p<0.001; ****, p<0.0001; (B) Tumors were removed at Day 7 from both groups to calculate final weight. Error bars represent s.d. p<0.0001; (C) Isolated tumors were processed for immunofluorescence with the stem cell marker, Sox2 (green) with the nuclei counterstained with DAPI (blue). Scale bars represent 10 μm.

FIGS. 5(A-D) illustrate that Eg5 inhibition improves survival in an orthotopic model; (A) GSCs from patient derived xenograft 3691 were pretreated for 18 hours with vehicle or 3 nM ispinesib. 2,500 or 25,000 viable cells were then intracranially (IC) injected (DMSO, n=10; ispinesib, n=7) into mice. Mice were then monitored for signs indicative of brain tumor development at which time they were sacrificed to generate a Kaplan-Meier survival curve. p=0.012 for the 2,500 cohort and p=0.020 for the 25,000 cohort, log-rank test; (B) Mice were subcutaneously injected in the flank with 100,000 bulk 3691 GBM cells. When tumors reached approximately 0.2-0.6 cm³, mice were randomized into one of two treatment groups; vehicle only (DMSO; n=5) or 10 mg/kg ispinesib (n=5) with daily administration over a 3 day time course. Tumors were then harvested and viable GSCs isolated. 1,000 or 10,000 viable cells were then intracranially (IC) injected into mice that were then monitored for signs indicative of brain tumor development at which time they were sacrificed to generate a Kaplan-Meier survival curve. p=0.0019 for the 1,000 cohort and p=0.021 for the 10,000 cohort, log-rank test; (C) A mouse bearing an orthotopic tumor was injected with a single dose of 10 mg/kg ispinesib. The tumor was isolated 5 hours later and processed for immunofluorescence to βtubulin with the nuclei counterstained with DAPI. Arrowheads indicate cells with monoastral spindles. a′ and a″ represent enlarged regions of interest; (D) 10,000 GSCs from patient derived xenograft 3691 modified to express luciferase were intracranial implanted into mice. 7 days later when positive luminescence signal indicated tumor burden, mice were randomized into one of two treatment groups; vehicle only (DMSO; n=10) or 10 mg/kg ispinesib (n=10) administered on a q4d×6 dosing schedule. Arrows indicate day of vehicle or drug administration. Mice were then monitored for signs indicative of brain tumor development at which time they were sacrificed to generate a Kaplan-Meier survival curve. p<0.001, log-rank test.

FIGS. 6(A-F) illustrate that Eg5 inhibition impacts cell motility; (A) Schematic of transwell assay. GSCs from patient derived xenograft 08-387 were enriched within interphase using a thymidine arrest and release paradigm. 125,000 interphase-enriched cells were plated per well of a matrigel coated transwell and given 8 hours to migrate in the presence of increasing concentrations of isinesib (0 to 200 nM) before fixation and staining of the nuclei with DAPI; (B) Resulting membranes were scored for the movement of nuclei through the transwell membrane. EC₅₀=67.2 nM. Error bars represent s.d.; (C) The transwell assay was run as above with vehicle (DMSO) or 200 nM ispinesib with data represented as the number of migrated cells per field. Error bars represent s.d. p=0.0009; (D) Schematic of slice culture assay. A PDGF-IRES-GFP retrovirus was used to generate tumors in a rat glioma model. Resulting tumor bearing brains were isolated to generate slice cultures. GFP-positive tumor cells were monitored by time-lapse video microscopy over a 10-hour time course; (E) wind-rose plots; (F) MSD graph.

FIGS. 7(A-F) illustrate that Eg5 plays a role in microtubule growth within the leading process; (A) Nascent cellular process formation was monitored for 6 hours via time-lapse microscopy in the presence of vehicle (DMSO) or ispinesib (200 nM) in acutely plated 08-387 GSCs enriched in interphase. Representative images of the two treatment groups are shown at the 6-hour time point. Cell bodies are masked in red and cell processes masked in yellow; (B) Process length was measured over the time course. Error bars represent s.d. p<0.0001; (C) Representative images from a modified scratch wound assay used to drive formation of a leading cellular process using 08-387 GSCs enriched in interphase. Just prior to wound formation, media was changed to that containing vehicle (DMSO) or ispinesib (200 nM). 6 hours later, cells were fixed and processed for immunofluorescence to β-tubulin; (D) The tubulin signal in the primary cell layer adjacent to the wound was quantified for both treatment groups and presented as signal over area; (E) Representative images of GSCs (08-387) expressing EB1-EGFP following 7 hour treatment with vehicle or 200 nM ispinesib; (F) Microtubule growth times were measured 30 minutes and 7 hours following drug treatments. Data are mean±SEM, calculated using the number of microtubules as sample size. Statistics were calculated using the Kruskal-Wallis test, with subsequent Dunn-Sidak test for multiple comparisons.

FIGS. 8(A-B) illustrate that Eg5 impacts microtubule growth through the CRMP-tubulin deposition pathway; (A) 08-387 GSCs enriched in interphase were exposed to vehicle (DMSO) or ispinesib (200 nM) and harvested for whole cell lysates every two hours over a 6 hour time course. Resulting lysates were probed for CRMP2 and pCRMP2 (T514); (B) Human brain microvascular endothelial cells (HuBrMVECs) were treated with vehicle (DMSO) or ispinesib (200 nM) and harvested for whole cell lysates every three hours over a 6 hour time course. Resulting lysates were probed for CRMP2 and pCRMP2 (T514); For all immunoblots, βactin served as a loading control. Molecular weight (MW) of resulting bands is given in kilodaltons (kD).

FIG. 9 is an illustration modeling the effect of an Eg5 inhibitor on microtubule polymerization associated with cell process extension in accordance with one aspect of the present invention.

### DETAILED DESCRIPTION OF THE INVENTION

This application relates to methods for using Eg5 inhibitors, and pharmaceutical compositions comprising the same, to treat glioblastoma multiforme (GBM) in subjects in need thereof. It has been shown that Eg5 protein is upregulated in GBM tumor cells compared to normal brain tissue and that the Eg5 protein is localized to the spindles of mitotically active GBM cells. It has been discovered that Eg5 is an essential driver of both the proliferative and invasive phenotypes that are characteristic of GBM generally and of the glioblastoma stem cell (GSC) subpopulation in particular. It has been further discovered that the malignant phenotype of glioblastoma can be blocked using an inhibitory agent of the mitotic kinesin Eg5.

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the application.

### DEFINITIONS

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

The invention is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

As used herein, the terms “treatment,” “treating,” or “treat” refer to any treatment of brain cancer, e.g., glioblastoma multiforme in a subject including, but not limited to, inhibiting disease development, arresting development of clinical symptoms associated with the disease, and/or relieving the symptoms associated with the disease. However, the terms “treating” and “ameliorating” are not necessarily meant to indicate a reversal or cessation of the disease process underlying the cancer afflicting the subject being treated. Such terms indicate that the deleterious signs and/or symptoms associated with the condition being treated are lessened or reduced, or the rate of progression or metastasis is reduced, compared to that which would occur in the absence of treatment. A change in a disease sign or symptom can be assessed at the level of the subject (e.g., the function or condition of the subject is assessed), or at a tissue or cellular level. In accordance with the present invention, desired mechanisms of treatment at the cellular level include, but are not limited to one or more of a reduction of cancer cell process extension and cell migration, apoptosis, cell cycle arrest, cellular differentiation, or DNA synthesis arrest.

As used herein, the term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those having an enhanced risk of developing precancers and cancers. An elevated risk represents an above-average risk that a subject will develop cancer, which can be determined, for example, through family history or the detection of genes causing a predisposition to developing cancer.

“Pharmaceutically acceptable” as used herein means that the compound or composition is suitable for administration to a subject to achieve the treatments described herein, without unduly deleterious side effects in light of the severity of the disease and necessity of the treatment.

As used herein, the term “effective amount” refers to an amount of an Eg5 inhibitor, particular a small molecule Eg5 inhibitor, which is sufficient to provide a desired effect. For example, a “therapeutically effective amount” provides an amount that is effective to reduce or arrest a disease or disorder such as abnormal cell growth or cell migration in a subject. The result can be a reduction and/or alleviation of the signs, symptoms, or causes of a disease or disorder, or any other desired alteration of a biological system. The effectiveness of treatment may be measured by evaluating a reduction in tumor load or decrease in tumor growth or tumor cell invasion and/or migration in a subject in response to the administration of an Eg5 inhibitor. The reduction in tumor load may be represent a direct decrease in mass, or it may be measured in terms of tumor growth delay, which is calculated by subtracting the average time for control tumors to grow over to a certain volume from the time required for treated tumors to grow to the same volume. The decrease in tumor cell metastasis may represent a direct decrease in tumor cell migration, or it may be measured in terms of the delay of tumor cell metastasis. An effective amount in any individual case may be determined by one of ordinary skill in the art using routine experimentation.

The term “subject” for purposes of treatment includes any human or animal subject who has a disorder characterized by unwanted, rapid cell proliferation. Such disorders include, but are not limited to cancers and precancers. In particular embodiments, the subject includes any human or animal subject that is suspected of having or has been diagnosed with GBM. For methods of prevention the subject is any human or animal subject, and preferably is a human subject who is at risk of acquiring a disorder characterized by unwanted, rapid cell proliferation, such as cancer. The subject may be at risk due to exposure to carcinogenic agents, being genetically predisposed to disorders characterized by unwanted, rapid cell proliferation, and so on. Besides being useful for human treatment, the compounds of the present invention are also useful for veterinary treatment of mammals, including companion animals and farm animals, such as, but not limited to dogs, cats, horses, cows, sheep, and pigs. Preferably, subject means a human.

As used herein, the terms “patient diagnosed with GBM”, “patient having GBM” or “patients identified with GBM” refers to patients that are identified as having or likely having GBM, a Grade IV astrocytoma. Nonlimiting examples of diagnosing a patient with GBM include diagnoses using histological analysis conducted by a board-certified pathologist and diagnostic tests based on molecular approaches.

The term “Eg5” refers to the kif11 gene product, a member in a class of kinesin-related proteins that are involved in functions related to movements of organelles, microtubules, or chromosomes along microtubules. These functions include axonal transport, microtubule sliding during nuclear fusion or division, and chromosome disjunction during meiosis and early mitosis. Eg5 appears to play a critical role in mitosis of mammalian cells. Sequences for Eg5 are set forth in, e.g., Genbank Accession Nos. NM_{—}004523 (human) and NM_{—}010615 (mouse).

The term “interfering RNA” or “RNAi” or “interfering RNA sequence” refers to double-stranded RNA (i.e., duplex RNA) that targets (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes “small-interfering RNA” or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini. The siRNA can be chemically synthesized or maybe encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem. 243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript.

### Eg5 Inhibitors

Eg5 inhibitors for use in a method of the present invention can include any agent which inhibits the Eg5 protein sufficiently to reduce or decrease the malignant phenotype of glioblastoma tumor cells in a subject. For example, suitable Eg5 inhibitors can inhibit mitotic progression of tumor cells as well as cell motility. In some embodiments, an Eg5 inhibitor for use in the present invention inhibits GBM tumor cell motility by disrupting the activity of the tubulin transport protein CRMP2 in a tumor cell.

Eg5 inhibitors for use in a method of the present invention can include small molecule inhibitors of Eg5. In some embodiments, an Eg5 inhibitor can include small molecules capable of targeting a motif in the catalytic domain of Eg5. In some embodiments, specific small molecule inhibitors of Eg5 exert their action through binding to an allosteric site located between a helix 3 and loop 5 of the Eg5 domain. The extended loop5 is found only in class 5 kinesins that is why these inhibitors are specific only to Eg5.

Eg5 inhibitors for use in the present invention can include those Eg5 inhibitors described in El-Nassan, European Journal of Medicinal Chemistry, 62:614-631 (2013), the disclosures of which is incorporated herein by reference. For example, El-Nassan describe suitable Eg5 inhibitors for use in the present invention including those Eg5 inhibitors from the dihydropyrimidine (DHPM), quinazoline, thiazolopyrimidine, Hexahydro-2H-pyrano[3,2-c]quinolones (HHPQ), thiadiazole, 4,5-dihyropyrazole, 2,4-diaryl-2,5-dihydropyrolle, dihydropyrazolobenzoxazine, isoquinolines, imidazoles, biphenyl, and benzimidazole chemical classes as well as naturally occurring Eg5 inhibitors.

Exemplary Eg5 inhibitors for use in the present invention can include, but are not limited to

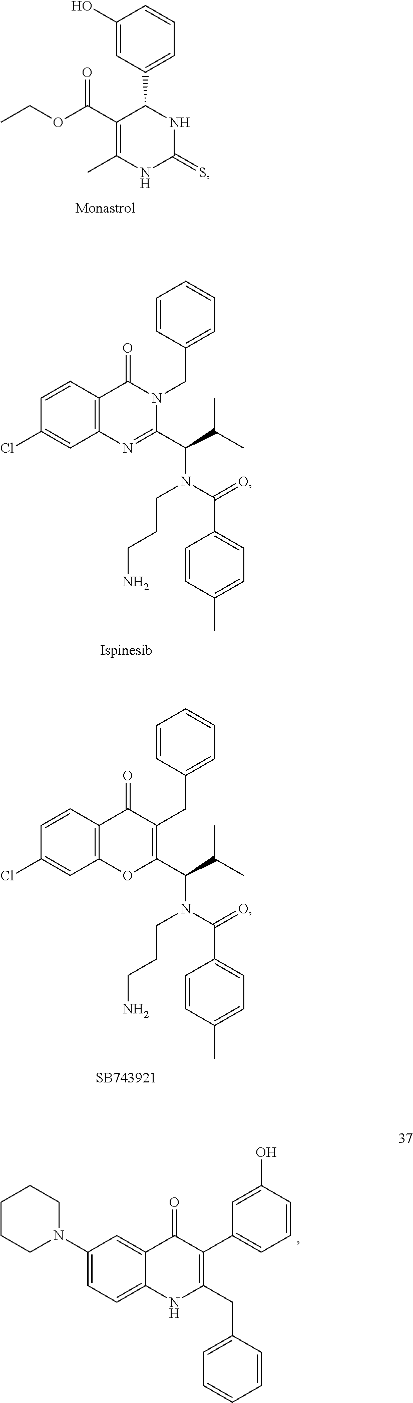 [SMILES: CCOC(=O)C1=C(C)NC(=S)N[C@H]1c1cccc(O)c1 ; Cc1ccc(C(=O)N(CCCN)[C@@H](c2nc3cc(Cl)ccc3c(=O)n2Cc2ccccc2)C(C)C)cc1 ; Cc1ccc(C(=O)N(CCCN)[C@@H](c2oc3cc(Cl)ccc3c(=O)c2Cc2ccccc2)C(C)C)cc1 ; O=c1c(-c2cccc(O)c2)c(Cc2ccccc2)[nH]c2ccc(N3CCCCC3)cc12]

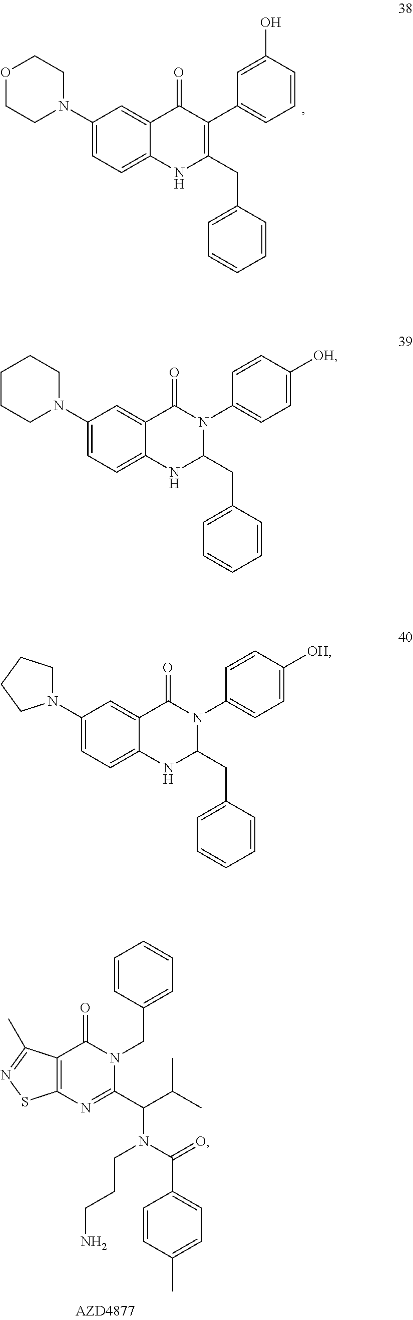 [SMILES: O=c1c(-c2cccc(O)c2)c(Cc2ccccc2)[nH]c2ccc(N3CCOCC3)cc12 ; O=C1c2cc(N3CCCCC3)ccc2NC(Cc2ccccc2)N1c1ccc(O)cc1 ; O=C1c2cc(N3CCCC3)ccc2NC(Cc2ccccc2)N1c1ccc(O)cc1 ; Cc1ccc(C(=O)N(CCCN)C(c2nc3snc(C)c3c(=O)n2Cc2ccccc2)C(C)C)cc1]

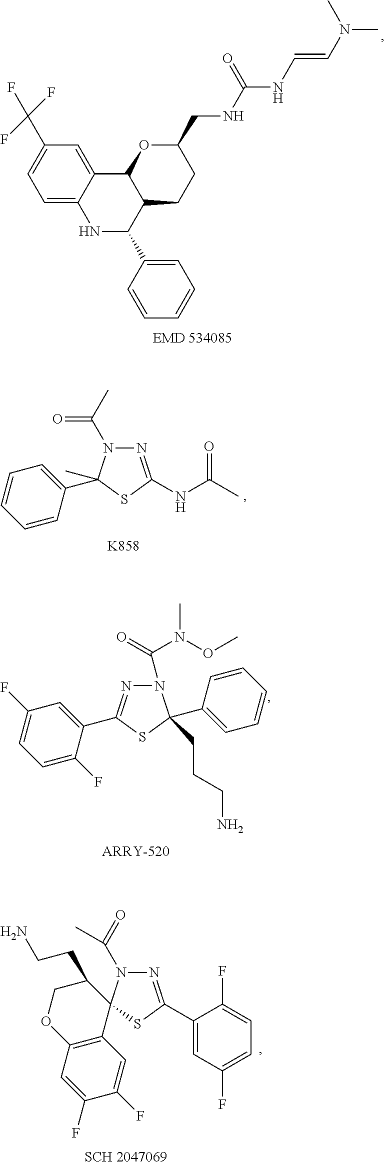

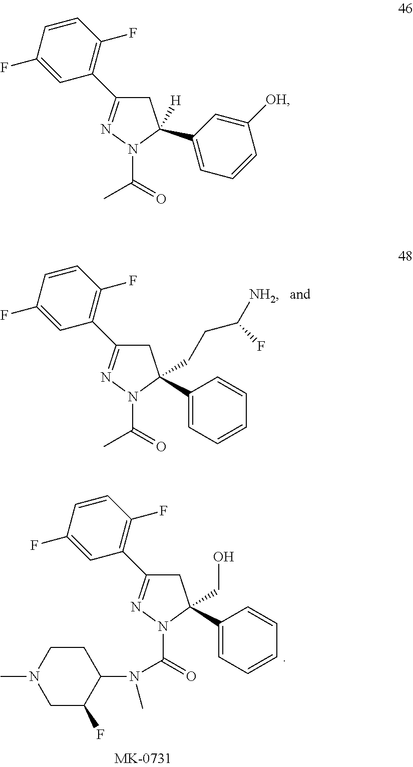 [SMILES: CC(=O)N1N=C(c2cc(F)ccc2F)C[C@H]1c1cccc(O)c1 ; CC(=O)N1N=C(c2cc(F)ccc2F)C[C@@]1(CC[C@H](N)F)c1ccccc1 ; "and" ; CN1CCC(N(C)C(=O)N2N=C(c3cc(F)ccc3F)C[C@@]2(CO)c2ccccc2)[C@@H](F)C1]

Eg5 inhibitors for use in the present invention can further include naturally occurring Eg5

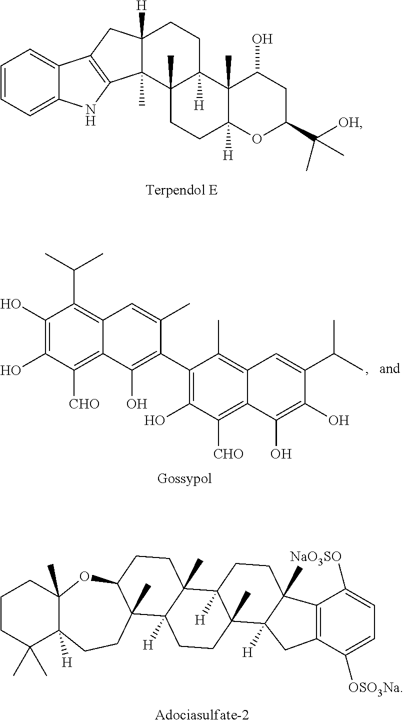

inhibitors such as, but not limited to,

In an exemplary embodiment, the Eg5 inhibitor includes the quinaloline derivative ispinesib, (N-(3-amino-propyl)-n-[R-1-(3-benzyl-7-choloro-4-oxo-3,4-dihydroquinazolin-2-yl)-2-methyl-propyl]-4-methyl-benzamide methanesulfonate) having the structure:

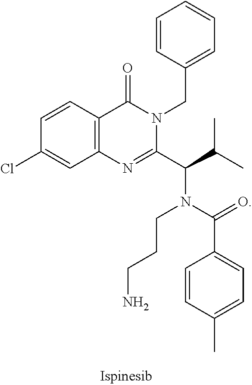 [SMILES: Cc1ccc(C(=O)N(CCCN)[C@@H](c2nc3cc(Cl)ccc3c(=O)n2Cc2ccccc2)C(C)C)cc1]

Eg5 inhibitors for use in the present invention can include non-small molecule Eg5 inhibitors. Non-small molecule Eg5 inhibitors can include but are not limited to Eg5 inhibitory nucleic acids or Eg5-specific inhibitory antibodies.

In some embodiments, the Eg5 inhibitory nucleic acid is a siRNA. siRNA is a duplex RNA which specifically cleaves target molecules to induce RNA interference (RNAi). Preferably, the siRNA of the present invention has a nucleotide sequence composed of a sense RNA strand homologous entirely or partially to a gene expressing an Eg5 protein nucleic acid sequence and an antisense RNA strand complementary thereto, which hybridizes with its target sequence within cells to silence Eg5 expression. An “effective amount” or “therapeutically effective amount” of a siRNA is an amount sufficient to produce the desired effect, e.g., a decrease in the expression of a target sequence in comparison to the normal expression level detected in the absence of the siRNA

In some embodiments, the Eg5 inhibitor can include Eg5-specific inhibitory antibodies. Exemplary inhibitory antibodies against Eg5 inhibitors of human mitotic kinesin Eg5 are described in Blangy A et al, Phosphorylation by p34cdc2 regulates spindle association of human Eg5, a kinesin-related motor essential for bipolar spindle formation in vivo. Cell 1995; 83:1159-69, the disclosures of which are incorporated herein by reference.

Candidate Eg5 inhibitory agents for use in a method of the present invention may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a particular agent inhibits Eg5 protein activity in the animal. More specifically, candidate agents can be used in these animal models to determine if a particular agent inhibits Eg5 protein activity in an animal model's brain tumor cells (i.e., GBM tumor cells). Alternatively, or in addition, candidate agents can be used in these animal models to determine if a candidate agent decreases one or more of the symptoms associated with the cancer, including, for instance, cancer cell proliferation, cancer metastasis, cancer cell motility, cancer cell invasiveness, or combinations thereof.

### Use of Eg5 Inhibitors for Brain Cancer Treatment

Eg5 inhibitors described herein can be used as a pharmaceutical. In particular they are useful for treating brain cancer, including glioblastoma multiforme (GBM). “Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

Brain cancer refers to an intracranial solid neoplasm or tumor found in the brain or the central spinal canal. They are created by an abnormal and uncontrolled cell division, usually in the brain itself, but also in lymphatic tissue, in blood vessels, in the cranial nerves, in the brain envelopes (meninges), skull, pituitary gland, or pineal gland. Tumors can be benign or malignant, can occur in different parts of the brain, and may or may not be primary tumors. A primary tumor is one that has started in the brain, as opposed to a metastatic tumor, which is something that has spread from one part of the body to another. The incidences of metastatic tumors are more prevalent than primary tumors by a ratio of 4:1. Tumors may or may not be symptomatic: some tumors are discovered because the patient has symptoms, others show up incidentally on an imaging scan, or at an autopsy. The most common primary brain tumors, listed in the order corresponding to their prevalence, are glioblastoma (GBM), meningiomas, pituitary adenomas, and nerve sheath tumors. Symptoms of brain tumors are well known to those skilled in the art. The preferred method for diagnosing a brain tumor is the use of imaging.

In one aspect of the invention, Eg5 inhibitors described herein can be used as a pharmaceutical in a method of treating GBM in a subject in need thereof. The method includes administering a therapeutically effective amount of an Eg5 inhibitor to the subject.

Glioblastoma involves glial cells, and include four subtypes, which are proneural, neural, mesenchymal, and classical glioblastoma. See Verhaak et al., Cancer Cell 17 (1): 98-110 (2010). Ninety-seven percent of tumors in the ‘classical’ subtype carry extra copies of the Epidermal growth factor receptor (EGFR) gene, and most have higher than normal expression of Epidermal growth factor receptor (EGFR), whereas the gene TP53, which is often mutated in glioblastoma, is rarely mutated in this subtype. In contrast, the proneural subtype often has high rates of alterations in TP53, and in PDGFRA, the gene encoding a-type platelet-derived growth factor receptor, and in IDH1, the gene encoding isocitrate dehydrogenase-1. The mesenchymal subtype is characterized by high rates of mutations or other alterations in NF1, the gene encoding Neurofibromatosis type 1 and fewer alterations in the EGFR gene and less expression of EGFR than other types. Many other genetic alterations have been described in glioblastoma, and the majority of them are clustered in three pathways, the P53, RB, and the PI3K/AKT.

In certain embodiments, Eg5 inhibitors described herein are useful for treating GBM in a subject, wherein the GBM is characterized by the presence of glioma cancer stem cells (GSCs). GSCs are phenotypically similar to the normal stem cells, they express CD133 gene and other genes characteristic of neural stem cells and posses the self-renewal potential. Cancer stem cells derived from glioblastoma are capable of recapitulating original polyclonal tumors when xenografted to nude mice. They are chemoresistant and radioresistant and therefore responsible for tumor progression and recurrence after conventional glioblastoma therapy. These GBM tumor initiating cells or GBM tumor propagating cells are functionally defined through assays of self-renewal and tumor propagation. See Reya et al., Nature, 414:105-11 (2001) and Hjelmeland et al., Nature Neuroscience, 14, 1375-81 (2011), the disclosures of which are incorporated herein by reference.

Suitable subjects benefiting from the methods of the present invention include male and female mammalian subjects, including humans, non-human primates, and non-primate mammals. Other suitable mammalian subjects include domesticated farm animals (e.g., cow, horse, pig) or pets (e.g., dog, cat). In some embodiments, the subject includes any human or animal subject who has a disorder characterized by unwanted, rapid cell proliferation of brain cells. Such disorders include, but are not limited to cancers and precancers, such as those including brain cancer, glioblastoma, and GBM characterized by the presence of glioma cancer stem cells. For methods of prevention the subject is any human or animal subject, and preferably is a human subject who is at risk of obtaining a disorder characterized by unwanted, rapid cell proliferation, such as cancer. The subject may be at risk due to exposure to carcinogenic agents, being genetically predisposed to disorders characterized by unwanted, rapid cell proliferation, and so on.

### Combination Therapies for Brain Cancer Treatment

The Eg5 inhibitors may be administered alone or in conjunction with other conventional treatments for brain cancer, as an adjunct therapy. An example of such adjuvant therapy is the additional treatment usually given after surgery to remove a tumor where all detectable disease has been removed, but where there remains a statistical risk of relapse due to occult disease.

The phrase “adjunct therapy”, “adjuvant therapy” or “combination therapy” in defining use of a Eg5 inhibitor described herein and one or more other pharmaceutical agents or conventional treatments for brain cancer (e.g., surgical resection, radiotherapy, chemotherapy, etc.), is intended to embrace administration of each therapy in a sequential manner in a regimen that will provide beneficial effects of the therapeutic combination, and is intended as well to embrace co-administration of therapeutic agents/surgical therapies in a substantially simultaneous manner, such as in a single formulation having a fixed ratio of active agents, or in multiple, separate administrations for each therapy.

As used herein the term “conventional treatment”, “standard treatment”, or “standard therapy” refers to the therapeutic treatment that is normally provided to patients diagnosed with a given condition. For the purpose of this application, a standard treatment for newly diagnosed glioblastoma multiforme (GBM) patients may include surgical resection followed by radiation and concurrent chemotherapy (e.g., cisplatin or temozolomide (TMZ)).

Pharmaceutical agent(s) administered to a subject in an adjunct therapy in combination with an Eg5 inhibitor can include antineoplastic agents or other growth inhibiting agents or other drugs or nutrients. For the purposes of a combination or adjuvant therapy, there are large numbers of antineoplastic agents available in commercial use, in clinical evaluation and in pre-clinical development, which could be selected for treatment of cancers or other disorders characterized by rapid proliferation of cells by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents. Suitable agents which may be used in combination therapy will be recognized by those of skill in the art.

In some embodiments, the antineoplastic agent(s) used in an adjunct therapy in combination with an Eg5 inhibitor are selected from those that have been approved for use in treating brain cancer. Examples of anticancer agents demonstrated to be effective against brain cancer include Everolimus, Bevacizumab, Procarbazine, Carmustine, Lomustine, and Temozolomide. However, in some embodiments, compounds such as Irinotecan, Cisplatin, Carboplatin, Methotrexate, Etoposide, Bleomycin, Vinblastine, Actinomycin, Cyclophosphamide, or Ifosfamide can be also be used to treat brain cancer.

In some embodiments, radioprotective agents known to those of skill in the art for radiotherapy may be administered in combination with an Eg5 inhibitor for the treatment of GBM in a subject. Radiotherapy may include ionizing radiation, particularly gamma radiation irradiated by commonly used linear accelerators or radionuclides. The radiotherapy to GBM tumors by radionuclides may be achieved externally or internally. Radiotherapy may include brachytherapy, radionuclide therapy, external beam radiation therapy, thermal therapy (cryoablation hyperthermia), radiosurgery, charged-particle radiotherapy, neutron radiotherapy and photodynamic therapy, and the like.

Radiotherapy may induce cell cycle delay or cell death through DNA damage in neoplastic cells by radiation to remove abnormal cells, but it has problems in that recurrence of cancer may be induced due to intrinsic radioresistance of cancer cells and the resulting increase in resistance to radiotherapy. In GBM, adjuvant chemoradiotherapy is critical in the case of a completely removed tumor, as with no other therapy, recurrence occurs in 1-3 months. Following surgical resection, residual GBM cells that have penetrated beyond the resection site may revert to a proliferative state to produce a more aggressive recurrent tumor that continues to disperse into nonneoplastic brain tissue and beyond.

Radiotherapy can be implemented by using a linear accelerator to irradiate the affected part with X-rays or an electron beam. While the X-ray conditions will differ depending on how far the tumor has advanced and its size and the like, a normal dose will be 1.5 to 3 Gy, preferably around 2 Gy, 2 to 5 times a week, and preferably 4 or 5 times a week, over a period of 1 to 5 weeks, for a total dose of 20 to 70 Gy, preferably 40 to 70 Gy, and more preferably 50 to 60 Gy. While the electron beam conditions will also differ depending on how far the tumor has advanced and its size and the like, a normal dose will be 2 to 5 Gy, preferably around 4 Gy, 1 to 5 times a week, and preferably 2 or 3 times a week, over a period of 1 to 5 weeks, for a total dose of 30 to 70 Gy, and preferably 40 to 60 Gy.

Therapeutic administration of Eg5 inhibitors described herein can also be combined with treatments such as hormonal therapy, proton therapy, cryosurgery, and high intensity focused ultrasound (HIFU), depending on the clinical scenario and desired outcome.

In certain embodiments, a therapeutic method described herein including the administration of an Eg5 inhibitor can be used to minimize GBM metastasis before, during or after a surgical procedure targeting GBM tumor cells. For example, a therapeutically effective amount of an Eg5 inhibitor can be administered to a subject before, during or after GBM tumor surgical resection surgery or brain stereotactic radiosurgery (i.e., gamma knife radiosurgery).

Eg5 inhibitors may be administered before conventional GBM treatment to a subject as a neoadjuvant therapy. For example, a therapeutically effective amount of an Eg5 inhibitor can be administered to a subject as a neoadjuvant therapy before GBM tumor surgical resection surgery or brain stereotactic radiosurgery. Neoadjuvant therapy, in contrast to adjuvant therapy, is given before the main treatment (e.g., surgery). For example, systemic Eg5 inhibitor therapy that is given before removal of a GBM tumor in a subject is considered neoadjuvant therapy. Neoadjuvant therapy using an Eg5 inhibitor can be employed to reduce the size of the tumor and/or minimize tumor cell invasion so as to facilitate more effective surgery. For example, the administration of an Eg5 inhibitor to a subject prior to the surgical resection or gamma knife treatment of a GBM tumor can minimize tumor cell invasion, thereby allowing for more complete removal of the tumor during surgery.

### Administration and Formulation of Eg5 Inhibitors

The medicament of the present invention can be one formulated for administration to a subject in need thereof with a pharmaceutically acceptable carrier. As examples of the pharmaceutically acceptable carrier, excipients such as sucrose, starch, mannitol, sorbitol, lactose, glucose, cellulose, talc, calcium phosphate, and calcium carbonate; binders such as cellulose, methylcellulose, hydroxylpropylcellulose, polypropylpyrrolidone, gelatin, gum arabic, polyethylene glycol, sucrose, and starch; disintegrants such as starch, carboxymethylcellulose, hydroxylpropylstarch, sodium-glycol-starch, sodium hydrogen carbonate, calcium phosphate, and calcium citrate; lubricants such as magnesium stearate, Aerosil, talc, and sodium lauryl sulfate; flavoring agents such as citric acid, menthol, glycyrrhizin-ammonium salt, glycine, and orange powder; preservatives such as sodium benzoate, sodium hydrogen sulfite, methylparaben, and propylparaben; stabilizers such as citric acid, sodium citrate, and acetic acid; suspending agents such as methylcellulose, polyvinylpyrrolidone, and aluminum stearate; dispersing agents such as surfactants; diluents such as water, physiological saline, and orange juice; base waxes such as cacao butter, polyethylene glycol, and kerosene; and the like can be mentioned, but these are not imitative.

The agents of the present invention are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

In some embodiments, an Eg5 inhibitor can be administered to a subject systemically, (i.e., enteral or parenteral administration). Preparations suitable for oral administration are a solution prepared by dissolving an effective amount of Eg5 inhibitor or a pharmaceutically acceptable salt thereof in a diluent such as water, physiological saline, or orange juice; capsules, sachets or tablets comprising an effective amount of Eg5 inhibitor in solid or granular form; a suspension prepared by suspending an effective amount of active ingredient in an appropriate dispersant; an emulsion prepared by dispersing and emulsifying a solution of an effective amount of active ingredient in an appropriate dispersant, and the like.

The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of compounds of formula I may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

Therapeutic agents described herein can be coated by a method known per se for the purpose of taste masking, enteric dissolution, sustained release and the like as necessary. As examples of coating agents used for the coating, hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudragit (manufactured by Rohm, Germany, methacrylic acid/acrylic acid copolymer), pigments (e.g., ferric oxide red, titanium dioxide lo and the like) and the like are used. The medicament may be a rapid-release preparation or sustained-release preparation. Examples of the base of the sustained-release preparation include liposome, atelocollagen, gelatin, hydroxyapatite, PLGA and the like.

As preparations suitable for parenteral administration (e.g., intravenous administration, subcutaneous administration, intramuscular administration, topical administration, intraperitoneal administration, intranasal administration, pulmonary administration and the like), aqueous and non-aqueous isotonic sterile injectable liquids are available, which may comprise an antioxidant, a buffer solution, a bacteriostatic agent, an isotonizing agent and the like. Aqueous and non-aqueous sterile suspensions can also be mentioned, which may comprise a suspending agent, a solubilizer, a thickener, a stabilizer, an antiseptic and the like. The preparation can be included in a container such as an ampoule or a vial in a unit dosage volume or in several divided doses. An active ingredient and a pharmaceutically acceptable carrier can also be freeze-dried and stored in a state that may be dissolved or suspended in an appropriate sterile vehicle just before use. In addition to liquid injections, inhalants and ointments are also acceptable. In the case of an inhalant, an active ingredient in a freeze-dried state is micronized and administered by inhalation using an appropriate inhalation device. An inhalant can be formulated as appropriate with a conventionally used surfactant, oil, seasoning, cyclodextrin or derivative thereof and the like as required. The Eg5 inhibitors may be incorporated into sustained-release preparations and devices.

The dosage of the medicament of the present invention varies depending on the kind and activity of active ingredient, seriousness of disease, animal species being the subject of administration, drug tolerability of the subject of administration, body weight, age and the like, and the usual dosage, based on the amount of active ingredient per day for an adult, can be about 0.0001 to about 100 mg/kg, for example, about 0.0001 to about 10 mg/kg, preferably about 0.005 to about 1 mg/kg. In certain embodiments, dosage can be about 10 mg/kg. The daily dosage can be administered, for example in regimens typical of 1-4 individual administration daily. Other preferred methods of administration include intraperitoneal administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

The present invention is illustrated by the following example. It is to be understood that the particular example, materials, amounts, and procedures are to be interpreted broadly in accordance with the scope and spirit of the invention as set forth herein.

### EXAMPLE

### Example 1

### The Malignant Phenotype of Glioblastoma can be Blocked with an Allosteric Inhibitor of the Mitotic Kinesin Eg5

### Experimental Procedures

### Animals and In Vivo Studies

All animal studies described were approved by the Cleveland Clinic Foundation Institutional Animal Care and Use Committee and conducted in accordance with the NIH Guide for the Care and Use of Laboratory Animals. For subcutaneous tumor studies, 1×10⁵ freshly dissociated GBM cells from a xenograft originally derived from a primary GBM patient specimen (3691) were injected into the flanks of 10 six week old female athymic nu/nu mice. Tumors were allowed to reach approximately 0.12 cm³, at which point animals were randomized into either vehicle control (DMSO) group or ispinesib (10 mg/kg given daily for 7 consecutive days by intraperitoneal injection) (Selleck Chemicals, Houston, Tex.). Tumors were monitored and measured daily using perpendicular diameter measurements for 7 days. Tumor volume was calculated using the ellipsoid formula π/6×larger diameter×(smaller diameter)². After the last treatment on Day 7, tumors were removed and weighed then processed for immunofluorescence (as described below). For intracranial implantation studies to evaluate tumor initiation by GSCs, the cells were isolated as described below and pre-exposed to drug or vehicle either in vitro or in vivo. For in vitro pre-exposure studies, GSCs from xenograft 3691 were seeded at single cell density and treated with vehicle or ispinesib (3 nM) for 18 hours (300,000 cells per condition). Drug was then washed off and 2,500 or 25,000 viable GSCs were implanted into the right frontal lobes of male NSG mice (n=10 for vehicle, n=7 for ispinesib). For in vivo pre-exposure studies, subcutaneous tumors were established as described above. When tumors reached ˜0.5 cm³, mice were randomized into a vehicle (DMSO) or ispinesib (10 mg/kg given daily for 3 consecutive days) group. 4 hours after the final treatment, GSCs were isolated as described below and 1,000 or 10,000 viable GSCs were then intracranially implanted into NSG mice (10 mice per group). Mice were monitored daily for neurological impairment at which time they were sacrificed and brains removed to evaluate for tumor development.

The PDGF-IRES-GFP retrovirus was produced according to well known methods previously described. P3 neonatal Sprague-Dawley rats were anesthetized using hypothermia. Briefly, each pup was carefully wrapped in several layers of gauze and then covered with ice. After 12 minutes, the rat pup's head was placed in a DKI 963 Stereotaxic Alignment Instrument (David Kopf Instruments, Tujunga, Calif.). The bregma was identified and using a sterile 18 gauge needle a small hole was poked into the skull that was 1 mm rostral and 2 mm to the right of the bregma. A 27-gauge Hamilton microsyringe (Reno, Nev.) was then inserted to a depth of 1.5 mm, and 2 ul of virus was injected at a rate of 0.2 ul/min.

### Isolation and Culture of GSCs and NSTCs

Human tissues were acquired from primary human brain tumor patient specimens in accordance with appropriate approved Institutional Review Board protocols. Tumor grade and available cytogenetic information for each specimen has been previously described. Tumor specimens were maintained through subcutaneous xenografts in the flanks of athymic Nu/Nu or NSG mice. Tumors were dissociated using a papain dissociation system (Worthington Biochemical, Lakewood, N.J.). GSCs were isolated based on surface expression of glycosylated CD133 enriched either by FACS (anti-CD133/2 (293C3)-APC, human) or magnetic activated cell sorting (anti-CD133/1 (AC133), human) as per manufacturers recommendations (MACS; Miltenyi Biotec, San Diego, Calif.) and grown as tumorspheres or adherently plated on GelTrex (Life Technologies, Grand Island, N.Y.). All cells were cultured at 37° C. in an atmosphere of 5% CO₂. GSCs were cultured in Neurobasal media (Life Technologies) with B27 (without Vitamin A; Life Technologies), basic fibroblast growth factor (bFGF, 10 ng/ml; R&D Systems, Minneapolis, Minn.), epidermal growth factor (EGF, 10 ng/ml; R&D Systems), L-glutamine (2 mM; Life Technologies), and sodium pyruvate (1 mM; Life Technologies). Freshly isolated NSTCs were grown in DMEM (Cleveland Clinic Media Productions Core, Cleveland, Ohio) supplemented with 10% fetal bovine serum (Life Technologies). After isolation, all cells were utilized in experiments in under 5 passages. For all comparative experiments involving GSCs and NSTCs the cells were grown in the same culture conditions of Neuralbasal media without growth factors. For cell counting prior to each experiment, a single cell suspension was achieved using TrypLE (Life Technologies).

### Tissue Microarray Immunohistochemistry and Survival Analysis

The glioma tissue microarray (TMA) and semiquantification of immunohistochemical data was performed as described previously (Gilbert et al., 2014). Briefly, three 2-mm diameter cores per deidentified tumor were obtained and semiquantified on a relative scale from 0 to 3, with 0=negative and 3=strongest. Results from all 3 cores were averaged together to produce a final score for a tumor.

### Tissue Microarray Immunohistochemistry and Survival Analysis

Briefly, deidentified tissue microarrays (TMAs) were constructed from gliomas. Three 2-mm diameter cores per tumor were obtained, with each core embedded in a separate TMA block. A total of 104 cases comprised the TMAs, including 9 nonneoplastic controls (cortical dysplasias), 9 grade II astrocytomas, 11 grade III astrocytomas, 12 anaplastic oligodendrogliomas, 16 grade II oligodendrogliomas, and 47 grade IV glioblastomas (GBMs). Each TMA core was semiquantified on a relative scale from 0 to 3, with 0=negative and 3=strongest. Results from all 3 cores were averaged together to produce a final score for a tumor. Results were plotted based on WHO grade and differences were calculated via one-way ANOVA with post-hoc Tukey's test. Survival data was obtained on each case, and the degree of expression was correlated with survival via Log-rank (Mantel-Cox) Tests.

For immunohistochemistry, 5 μm TMA slides were baked at 60° C. for one hour, followed by deparaffinization in xylene and stepwise hydration in alcohol to TBS-Tween. Endogenous peroxidases were quenched with 3% hydrogen peroxide for 5 minutes and antigen retrieval was performed with Dako's high pH antigen retrieval buffer by heating to 110° C. for 20 minutes in a Biocare medical decloaking chamber followed by cooling to room temperature. Slides were blocked in 5% normal goat serum in TBST for 20 minutes and incubated in anti-Eg5 primary antibody (BD Biosciences, San Jose, Calif.) for 1 hour at room temperature. After washing in TBST, rabbit secondary antibody was applied for 30 minutes at room temperature (Dako Envision+ kit) followed by TBST washes and detection with DAB. Slides were then counterstained in Mayer's hematoxylin for 5 minutes and blued in ammonia water before dehydrating and cover slipping.

### Quantitative RT-PCR

Total RNA was isolated using the RNeasy isolation kit (Qiagen) and reverse transcribed into cDNA using the qScript cDNA SuperMix (QuantaBiosciences). mRNA was labeled with SYBR Green MasterMix (Applied Biosystems) and levels of mRNA measured on an Applied Biosystems 7900HT cycler using. Expression values were normalized to f3-Actin or GAPDH.

### Immunoblotting

Protein extracts were made using a 50 mM Tris pH 8.0, 120 nM NaCl, 0.5% NP-40 lysis solution. Resulting lysates were ran on 10% SDS-PAGE gels and transferred to Immobilon-FL PVDF (Millipore Corp., Billerica, Mass.). The membranes were blocked with 5% (wt/vol) bovine serum albumin in PBS-Tween-20 (0.2% vol/vol) and probed with primary antibodies against Eg5 (mouse, 1:4000, BD Biosciences, San Jose, Calif.), Olig2 (rabbit, 1:1000, abcam, San Francisco, Calif.), GFAP (DAKO; 1:10,000, Carpinteria, Calif.), CRMP2 (mouse, 1:1000, abgent, San Diego, Calif.), pCRMP2(T514) (rabbit, 1:1000, Cell Signaling, Beverly, Mass.), or β-actin (mouse, 1:8000, Sigma-Aldrich, St. Louis, Mo.) as a loading control. Secondary antibodies (LI-COR) were incubated in TBST+0.02% SDS and visualized with the LI-COR Odyssey near infrared imaging system (Lincoln, NB).

### Cell Synchronization and APC/C Assay

GSCs and NSTCs were plated adherently on GelTrex membrane (Gibco®, Life Technologies, Carslbad, Calif.). Synchronization at G1/S was achieved by exposing the cells to 2 mM thymidine for 18 hours, followed by washes with PBS and a release in fresh media for 8 hours, followed by a second exposure to 2 mM thymidine and a final wash and release in fresh media. Validation of synchronization was achieved via flow cytometry using propidium iodide.

### Dose Response and Cell Viability Assays

The half maximal effective concentration of ispinesib for acutely dissociated and MACS sorted matched GSCs and NSTCs was determined by plating, in triplicate, 1,000 cells per well of a 96 well plate pre-coated with GelTrex in growth-factor free Neuralbasal media. The next day, cells were exposed to 2-fold increasing concentrations of ispinesib spanning from 0 to 32 nM. Viability was measured at 72 hours using CellTiter-Glo ATP-based assay (Promega, Madison, Wis.) read on a luminometer (Perkin-Elmer, Waltham, Mass.). For long-term viability assays, 500 cells per well were plated in triplicate into 96-well plates as above. The next day, cells were exposed to 3 nM ispinesib or vehicle (DMSO) and viability was measured daily for 5 days using CellTiter-Glo. Day 0 reading were taken immediately after addition of drug or vehicle. SubG1 for GSCs and NSTCs was determined by flow cytometry for propidium iodide and gated as the events below the 2N G1 peak of the resulting histogram. Analysis was done following treatment of 100,000 cells with vehicle or 3 nM ispinesib. Cells were fixed at Day 0 through Day 4 in 100% cold ethanol and simultaneously processed for flow analysis with 20,000 events analyzed per condition.

### Immunofluorescence Imaging

Subcutaneous or intracranial tumors were fixed overnight in 4% PFA at 4° C., washed in PBS, cryoprotected in 30% sucrose, then embedded in optimal cutting temperature compound (OCT) and stored at −80° C. (Sakura Finetek USA, Torrance, Calif.). Sections were cut using a LeicaCMXXXX cryostat at 10 μm directly onto slides (Leica Microsystems Inc., Buffalo Grove, Ill.). Sections were postfixed for 15 minutes at room temperature in 4% PFA followed by PBS washes then incubated in blocking solution (5% goat serum, 0.1% Triton X-100 in PBS). Primary antibodies were diluted in blocking solution and used as follows overnight at 4° C.: Sox2 (rabbit, 1:500, Abcam, Cambridge, Mass., USA), atubulin (rat, 1:1000, AbD Serotec, Raleigh, N.C.). Secondary antibodies were Alexa Fluor (1:500, Invitrogen, Grand Island, N.Y., USA) labeled with 488 mixed in blocking solution and incubated for 2 hours at room temperature. DNA was stained using 4′,6-diamidino-2-phenylindole (DAPI, 10 μg/ml, Sigma, St Louis, Mo., USA) diluted in PBS for 5 minutes at room temperature.

### Transwell In Vitro Invasion Assay

08-387 GSCs enriched in G1 (8 hours post final thymidine release) were pre-exposed to vehicle or ispinesib (200 to 0 nM at a two-fold dilution) for one hour prior to being seeded onto Matrigel coated FluoroBlok Cell Culture Inserts at 125,000 cells per insert (±vehicle or ispinesib, plated in triplicate) with Neurobasal media supplemented with 10% FBS in the lower reservoir (BD Falcon, Franklin Lakes, N.J.). After 8 hours, membranes were processed for analysis with nuclei stained with DAPI. Nuclei on the lower surface of the membrane within the central field were scored using the 20× objective on a Leica DMIRB inverted microscopes.

### Nascent Process Analysis

08-387 GSCs enriched in interphase were seeded at 6,000 cells per well of a 6 well plate and allowed to adhere for 1 hour. Cells were then exposed to vehicle or 200 nM ispinesib and images recorded over a 6-hour period using the IncuCyte ZOOM (Essen Biosciences, Ann Arbor, Mich.). Process formation was measured using CellPlayer NeuroTrack Software Module (Essen Biosciences).

### Wound Assay

Interphase enriched 08-387 GSCs were seeded within two-well culture inserts (ibidi, Verona, Wis.) at 2×10⁵ cells per ml in triplicate (final volume of 80 μl per well) onto Geltrex coated glass bottom 6 well pates (MatTek Corp., Ashland, Mass.). After 2 hours, the insert was removed and cells were exposed to vehicle or 200 nM ispinesib. 6 hours later, cells were fixed and processed for immunofluorescence as described for atubulin. 20 Z-stack images at the wound edge were captured per condition using the 40× objective on a Leica DMI6000 inverted microscope. Images were then exported aa tif files using Velocity software (Mountain View, Calif.) and analyzed in ImageJ (NIH, Bethesda, Md.). Regions of interest were drawn for the primary cell layer at the wound edge for 3 independent scans per Z-stack representing the top, middle and bottom portion of the cell layer and pixel intensity and area measured.

### Endothelial Cell Experiment

Primary Human Brain Microvascular Endothelial Cells were purchased from Cell Systems (Kirkland, Wash.) and used at passages 3 through 6. Cells were maintained in EGM-2 MV media from Lonza (Walkersville, Md.) and stained with CellTracker™ Green CMFDA Dye from Invitrogen (Eugene, Oreg.) for fluorescent imaging. Tube formation assays were performed on Matrigel from Corning (Tewksbury, Mass.) in glass bottom 6-well plates from MatTek (Ashland, Mass.). Cells were plated at a density of 1.3×10⁴ per cm² and imaged at the indicated times.

### Measurement of Microtubule Dynamics

GSCs from patient derived xenograft 08-387 were cultured in Neurobasal Media, as previously described. Cells were plated on tissue culture dishes coated with GelTrex (Gibco®, Life Technologies, Carslbad, Calif.) and transfected with EB1-EGFP plasmid using FuGene® HD transfection reagent (Promega Corporation, Madison, Wis.) in a 3:1 (FuGene:DNA) ratio. Cells were transferred to GelTrex-coated 35 mm No. 1.5 glass MatTek dishes (MatTek Corporation, Ashland, Mass.) at a density of 50,000 cells per dish 48 hours following transfection, and allowed to adhere overnight prior to imaging. Dynamics measurements were obtained at 30 minutes and 7 hours following addition of vehicle (DMSO) or ispinesib (200 nM) to cell culture media.

Streaming time-lapse images of EB1-EGFP-expressing cells were acquired at 300 ms per frame for 1 minute (200 total frames) at using a Nikon TiE epifluorescence microscope (Nikon Instruments Inc., Melville, N.Y.) equipped with a Zyla 5.5 sCMOS camera (Andor Technology Ltd., Belfast, Ireland) under control of NIS Elements software (Nikon). Fluorescence illumination during imaging was provided by a SpectraX Light Engine® (Lumencor Inc., Beaverton, Oreg.) and a GFP/mCherry filter set (Chroma Technology Corp., Bellows Falls, Vt.). A 100×1.49 NA Apo TIRF oil immersion objective and 1.5× intermediate projection lens (150× total magnification) result in a spatial sampling of 42 nm/pixel for the imaging system. Cells were maintained at 37° C. and 5% CO₂ during imaging using a BoldLine stagetop incubation system (Okolab S.R.L., Ottaviano, Italy).

Microtubule growth rates and times were obtained from streaming timelapse images in MATLAB (MathWorks Inc., Natick, Mass.) using TipTracker (Demchouk et al., CMBE, 2011; Prahl et al., Meth Enzymol, 2014) in EB1-tracking mode (Seetapun et al., Curr Biol, 2012) without modification from the version current as of Apr. 27, 2014. Growth times were defined as the difference in time between the appearance and disappearance of EB1-EGFP signal for each individual microtubule. EB1 comet velocities were defined as the linear best-fit slope to the microtubule tip position to all frames where EGFP signal was visible. In some cases, EGFP signal would disappear then reappear within the same region, suggesting re-establishment of microtubule growth; these events were counted as separate growth episodes for the purposes of calculating growth times and comet velocities. Data were recorded for each condition and reported as mean±SEM (calculated using the number of microtubules). Statistical comparisons between groups were made using the Kruskal-Wallis test, with a subsequent Dunn-Sidak test for multiple comparisons between groups.

### Time-Lapse Microscopy

At days P8, P9, and P10, PDGF-IRES-GFP injected rat pups were anesthetized with Ketamine/Xylazine cocktail and decapitated. Brains were isolated and 300-um coronal sections were taken in the vicinity of the injection site using a McIlwain tissue chopper. The slices were transferred onto a porous 0.4-um culture plate insert (Millipore, Billerica, Mass.) and then placed into Glass Bottom 6 well plates (P06G-0-20-F, MatTek Corp, Ashland, Mass.) containing slice culture media (MEM, 2 mM L-glutamine, 10% NaCl, 10% Glucose, Its (Sigma, 12521) and Anti-Anti (Gibco, 15240-062)). Microglial and perivascular cells of the brain sections were stained with Isolectin GS-IB4, Alexa fluor 594 conjugate (Invitrogen) for 30 minutes at 37° C., washed three times with PBS and returned to a humidified atmosphere at 5% (CO₂). Images of the brain slices for time-lapse microscopy were acquired with a Perkin Elmer UltraView Spinning Disk microscope on a Leica DMI 6000 with a 10× Plan Apo 0.4 NA objective lens. During imaging, the brain slices were placed in a stage mounted 37° C. incubator where 60% humidity and 5% CO₂ was maintained. The emission range for 488 and 561 is 550-550 and 580-650, respectively and acquisition and analysis were performed using Velocity software. The migratory path of the individual cells was then tracked every 7 minutes by marking the position of the cell body at consecutive time points.

### Cell Migration Analysis

Fluorescence, time-lapse image stacks consisting of ˜150 frames (7 minute intervals) were imported into Image-Pro Plus (v7.0, Media Cybernetics, Silver Spring, Md.). Gliomal cell tracking for each treatment group (55 cells/group) was performed in a semi-automated fashion using a combination of Fourier correlation and user-guided input. Cells selected for tracking were chosen randomly from the periphery of the tumor mass in each image stack. X and Y coordinates from each cell track were exported to Excel and translated such that each cell's track originated from a common origin (0,0). The resulting data was displayed in a Wind Rose plot for each treatment group. Mean-squared displacement for tracked cells in each group was analyzed using a persistent random-walk model (1,2). Speed (um/s) and persistence (min) were extracted using a non-linear least-squared regression to the model equation for each track and averaged for each treatment group.

### Limiting Dilution Assay and Sphere Formation

GSCs were isolated from subcutaneous flank xenografts of patient-derived specimens by magnetic sorting as described above and allowed to recover overnight. The next day cells were exposed to vehicle or 3 nM ispinesib for 18 hours. Cells were then washed and triturated into a single cell suspension. Cell sorting was performed using a FACS Aria II Cell Sorter (BD Biosciences, Franklin Lakes, N.J., USA) to plate the cells into 96 well plates at a final cell number per well of 1 (38 wells/plate), 5 (24 wells/plate), 10, 20, or 50 (all at 12 wells/plate). Tumorsphere formation was evaluated 10 days after sorting and wells were scored positive or negative for the presence of at least one tumorsphere. The estimated stem cell frequency was calculated using extreme limiting dilution analysis. Only live cells were selected for using Live/Dead dye blue (Life Technologies).

### Phospho-Array

08-387 GSCs were enriched in G1 as described. Cells were then exposed to vehicle or ispinesib (200 nM) for 7 hours. Following treatment, cells were pelleted and snap frozen in liquid nitrogen before shipping to Full Moon Biosystems for processing to probe a Cytoskeleton Phospho Antibody Array containing 141 highly specific cytoskeleton related antibodies (Sunnyvale, Calif.).

### Statistical Analysis

Statistical significance was calculated with GraphPad Prism Software utilizing a 1-way or 2-way ANOVA with a Bonferroni's post-test, Student's t-test, or log-rank (Mantel-Cox) test where appropriate (GraphPad Software Inc.). Data are represented as the mean±standard deviation (s.d.).

### Results

### Mitotic Kinesins, Including Eg5, are Upregulated in GBM

GBMs are among the most proliferative of tumors, with a high mitotic index as one of its defining characteristics. As proliferation depends on mitotic kinesins, we sought to characterize the degree of expression of these mitotic regulators in human GBMs.

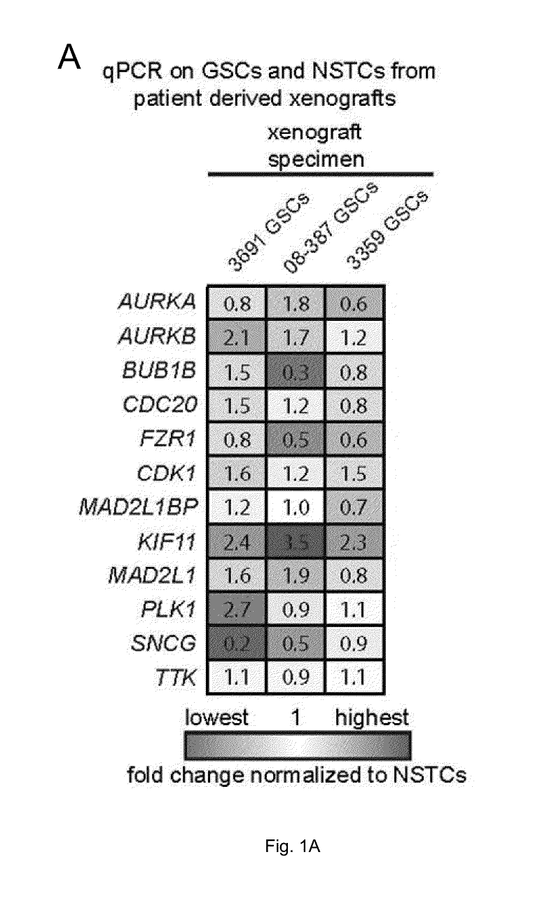

We also compared three matched sets of GSCs and NSTCs, from patient derived xenografts, for the differential expression of a group of mitotic regulators using quantitative-PCR. Of these, only Eg5 was consistently elevated above 2-fold (FIG. 1A). These GSCs were functionally validated for their ability to self-renew and form tumors in immunocompromised mice and express the GSC marker CD133. Eg5 is a particularly attractive target as amongst the mitotic kinesins it has moved the furthest in clinical trials. To confirm a correlation between Eg5 and glioma malignancy at the protein level, we probed a well-annotated tissue micro-array for Eg5 and found that Eg5 levels increased with WHO grade (p=0.0001; FIG. 1B). High Eg5 levels also significantly correlated with patient survival in high-grade glioma (p=0.047; FIG. 1C). Within GBM (WHO grade IV), Eg5 protein levels were markedly elevated over normal brain, with the specificity of the Eg5 antibody validated via localization to the spindles of mitotically active cells (FIGS. 1D and 1E). Together, these data establish that mitotic kinesins are consistently upregulated in GBM and they implicate Eg5 as a putative therapeutic target.

### Eg5 Protein Levels are Elevated in GSCs Due to Attenuated Protein Turnover

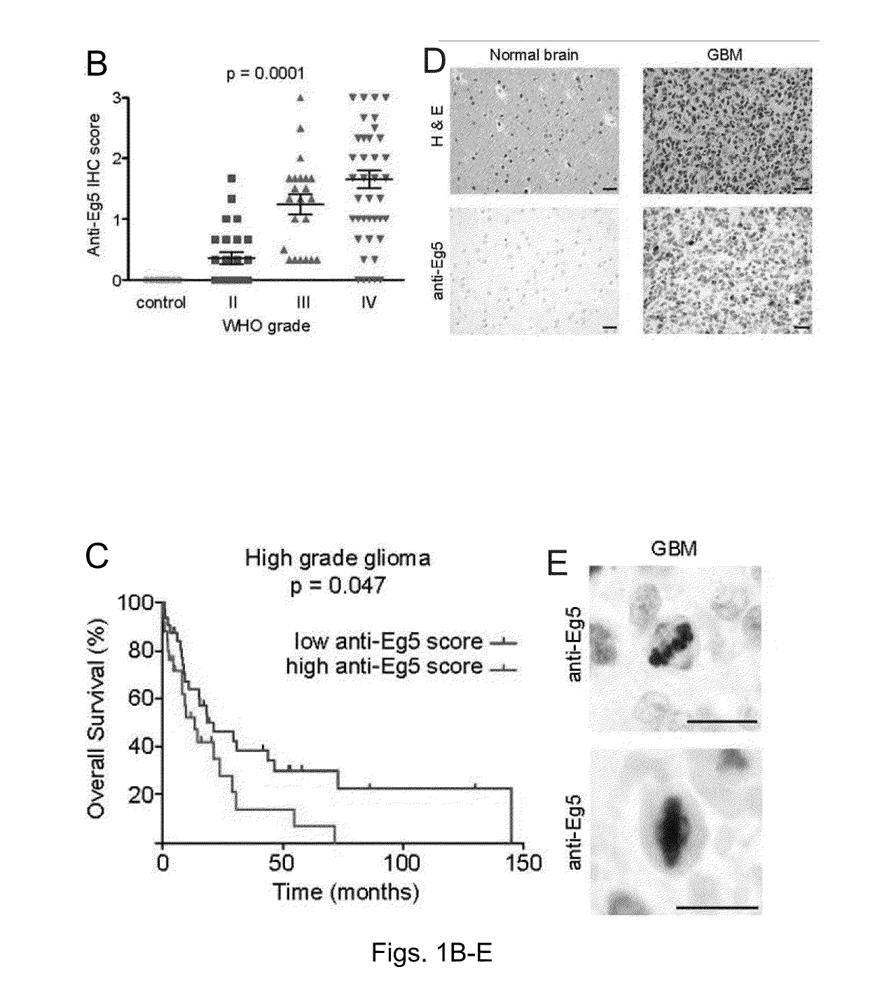

While the results described above demonstrate upregulation of Eg5 at the transcriptional level between GSCs and NSTCs, and at the protein level in GBMs in general, they do not indicate whether GSCs and NSTCs differ in their expression of Eg5 at the protein level. We therefore isolated matched GSCs and NSTCs from patient derived xenografts and evaluated Eg5 levels by Western blot. In all three specimens tested, Eg5 protein levels were higher in the GSCs (FIG. 2A). To validate the purity of our GSC and NSTC segregation we probed for Olig2 (marker for GSCs) and GFAP (marker for more differentiated NSTCs) with a higher level of Olig2 protein in the GSCs and corresponding decrease in the NSTCs and a higher level of GFAP protein in the NSTCs and a corresponding decrease in the GSCs (FIG. 2A).

In non-transformed cells, levels of Eg5 fluctuate throughout the cell cycle and are regulated by targeted protein destruction (Eguren et al., Cell Reports, 2014; Singh et al, EMBO, 2014) by means of ubiquitination and subsequent proteasome-mediated degradation. The anaphase promoting complex or cyclosome (APC/C) is an E3 ligase that is a central mediator of the ubiquitin-mediated degradation of mitotic proteins. APC/C substrate specificity is dictated by its association with the activator/substrate adaptors Cdc20 (APC/C^{Cdc20}; active in early mitosis) or Cdh1 (APC/C^{Cdh1}; active in late mitosis and during G1). Eg5 is ubiquitinated and degraded in an APC^{Cdh1}-dependent manner to reduce its levels in G1 (Eguren et al., Cell Reports, 2014). To determine if Eg5 turnover differs between GSCs and NSTCs and if this contributes to the differences in protein levels, we utilized a double-thymidine block to arrest GSCs and matched NSTCs from two GBM xenograft specimens. The cells were then released from the arrest and samples were harvested for protein lysates every two hours over a 10-hour time course for specimen 3691 and a 24-hour time course for specimen 08-387 and the resulting lysates were probed for Eg5 by Western blot. In addition, flow cytometry was performed to validate the degree of cell synchronization. Whereas Eg5 levels dropped in NSTCs following mitosis, as previously reported for non-transformed cells, GSCs maintained high levels of Eg5 throughout the cell cycle (FIGS. 2B and 2C). Since APC^{Cdh1} targets Eg5 for destruction, we also examined the levels of an additional APC^{Cdh1} target, Cdc20. Akin to Eg5, levels of Cdc20 failed to drop in G1 in GSCs. These results imply that APC^{Cdh1}, a central component in cell cycle regulation and tumor suppression, is dysfunctional in GSCs. To gain additional insight, we treated GSCs and NSTCs with nocodazole to arrest cells at the start of mitosis. Mitotically arrested cells were then released from the nocodazole block. Four hours later, at a time when the cells were in late mitosis/G1 and where APC^{Cdh1} should be active, extracts were generated. The activity of APC^{Cdh1} was directly tested by introducing hemagglutinin (HA)-tagged Securin, an APC^{Cdh1} substrate, into the lysates. The levels of HA-Securin were evaluated over time by Western blot to monitor the extent of protein turnover during the time course between GSCs and NSTCs. HA-Securin exhibited greater stability in extracts of G1 GSCs indicating that the activity of APC^{Cdh1} is attenuated in GSCs compared to NSTCs (FIG. 2D). These results indicate that chronic expression of central cell cycle regulators such as Eg5 and Cdc20 is a key feature in GSCs and establishes attenuated ubiquitin-mediated proteolysis as a contributing factor to the differential expression between GSCs and NSTCs.

Eg5 Inhibition Targets GSCs and Compromises their Ability to Self-Renew

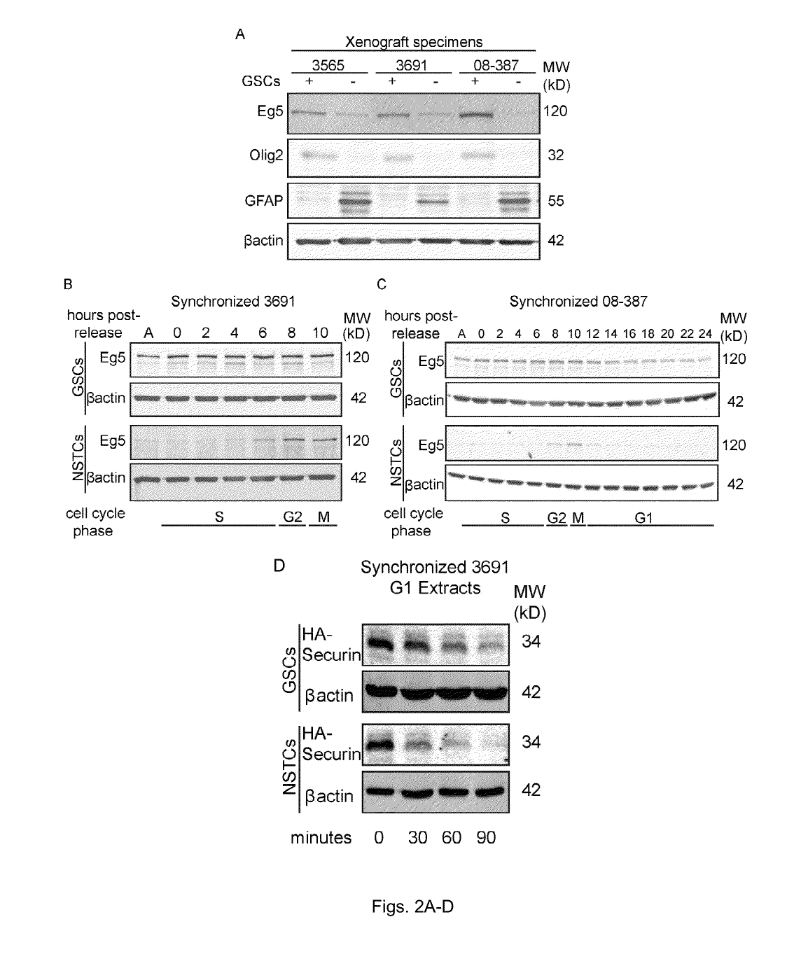

Highly specific Eg5 inhibitors have never been studied in GBM or in the therapy resistant GSC sub-population. To explore this, we utilized ispinesib, a cell permeable small molecule inhibitor to Eg5 with nanomolar efficacy in cell lines tested and 40,000 times more selectivity for Eg5 over other kinesins (Johnson et al, Am Assoc Cancer Res. 2002). GSCs and NSTCs were grown in the same culture conditions for all comparative experiments. We have previously used this approach to maintain the GSC or NSTC phenotypes while also providing mitogenic signals that produce similar cell cycle profiles and proliferation rates. We first determined the dose response relationships for cell kill versus ispinesib concentration for matched GSCs and NSTCs from 5 independent xenograft specimens. GSCs (EC₅₀=1.15±0.35 nM) had a slight but significant increased sensitivity to ispinesib over NSCTs (EC₅₀=1.79±0.22 nM) although both cell populations were sensitive to the drug in the nanomolar range (FIG. 3A). To further evaluate the impact of Eg5 inhibition on GSCs and NSTCs, cells were exposed for 96 hours to 3 nM ispinesib and then evaluated by flow cytometry to measure the subG1 population, a surrogate marker for apoptosis. GSCs demonstrated a higher subG1 fraction over NSTCs in the three xenograft patient specimens over the four days of observation (FIG. 3B). To confirm these findings, an ATP-based viability assay was used to monitor the impact of exposure to 3 nM ispinesib over a five-day time course for two matched GSCs and NSTCs. Both populations demonstrated sensitivity to Eg5 inhibition with the GSCs showing a slight increased sensitivity over the NSTCs at earlier time points (FIG. 3C). These data indicate that although GSCs and NSTCs have differential death kinetics, the viability of both cell populations is compromised following inhibition of Eg5.

To further investigate the impact of Eg5 inhibition on GSCs, we examined the effect of transient Eg5 inhibition on GSC self-renewal as measured by the ability of a single cell to form a tumorsphere in vitro. This can be quantified using a limiting dilution assay that permits estimation of stem cell frequency when the cells are plated in a range down to 1 cell/well, and later assayed for the presence or absence of tumorspheres. We sorted GSCs from 3 different patient-derived xenografts (3 independent tumors per specimen), and allowed them to recover overnight before pretreatment for 18 hours with 3 nM ispinesib or vehicle control. Cells were then released from drug inhibition and plated at single-cell densities using flow cytometry at a range of 1 cell per well to 50 cells per well and scored for tumorsphere formation ten days later. In all specimens tested, stem cell frequency was significantly compromised in GSCs pre-exposed to ispinesib. Together these data support Eg5 as a robust target for both GSCs and NSTCs with the ability to also impact GSC self-renewal.

### Eg5 Inhibition Impacts GSCs In Vivo

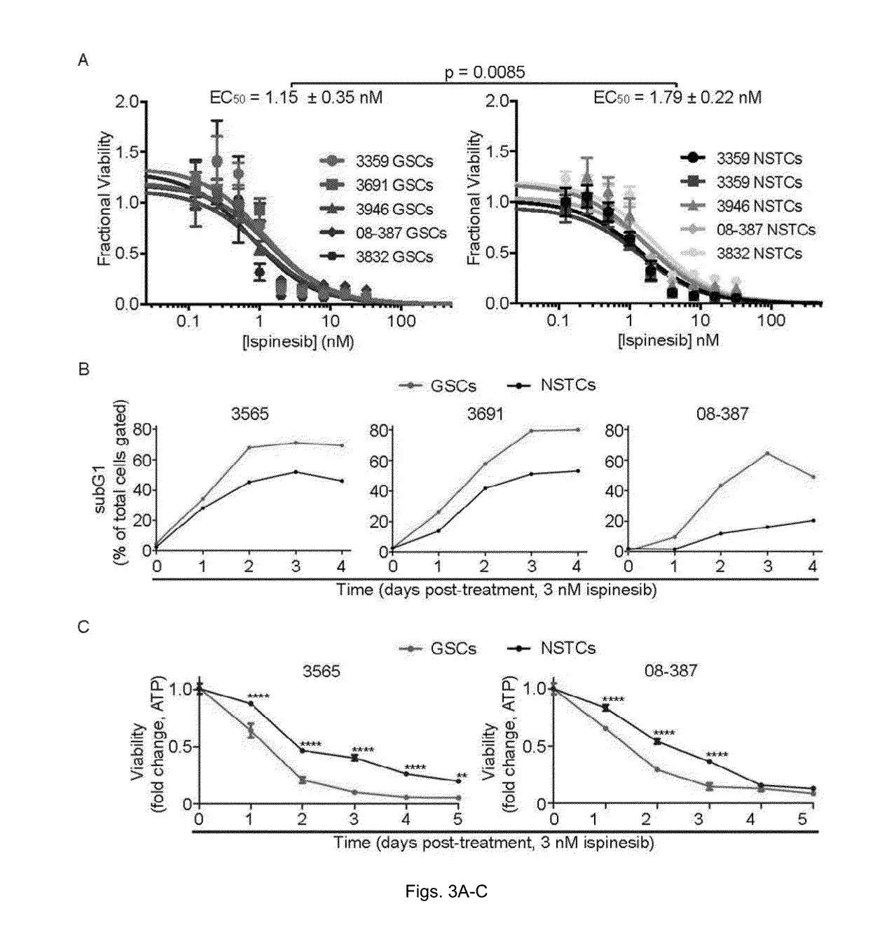

We next wanted to evaluate the impact of Eg5 inhibition on GSC propagation and tumor initiation in vivo. To address this, we utilized several approaches. First, we used a flank tumor model to test drug efficacy under conditions where the blood brain barrier would not interfere with drug delivery and where tumor isolation for evaluation of the GSC subpopulation could be readily performed. We utilized a 7-day inhibitor study to monitor the impact of acute in vivo exposure of drug on the GSC subpopulation. GBM cells (100,000/mouse) were injected into the flanks of NSG mice. When tumors reached 0.12 cm³, mice were randomized into either vehicle (DMSO) or ispinesib (10 mg/kg) treated groups with drug or vehicle given daily via intraperitoneal administered for 7 consecutive days. Flank tumor volume was measured daily. Two hours after the last administration (day 7), tumor weight and volume were measured. Ispinesib treatment significantly reduced tumor volume and weight (FIGS. 4A and 4B). We then evaluated the end stage tumors by immunocytochemistry for the stem cell marker Sox2. As FIG. 4C demonstrates, ispinesib treated tumors showed no Sox2 positive cells. These findings support in vivo cell kill of GBM cells including the GSC subpopulation.

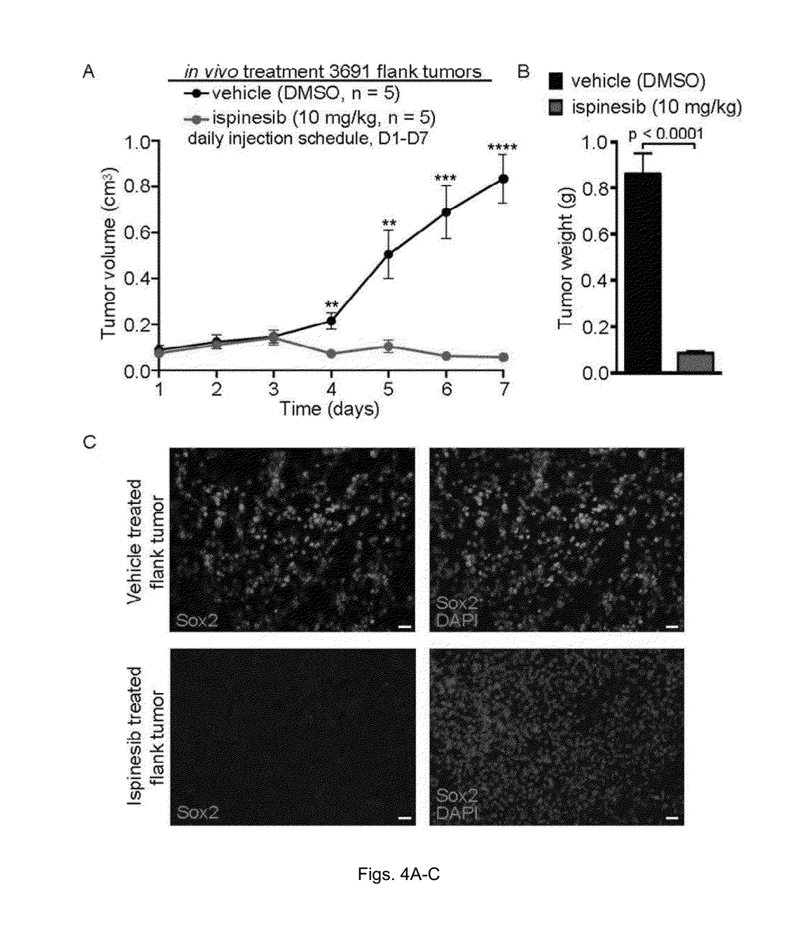

Tumor initiation is a defining characteristic of GSCs. To determine whether ispinesib treatment alters tumor initiation and survival in orthotopic xenograft models, we pretreated GSCs in vitro for 18 hours with either 3 nM ispinesib or an equal volume of vehicle (DMSO). Cells were then intracranially implanted at 2,500 or 25,000 cells per mouse (n=5 mice per group). Mice were monitored daily for weight loss and neurological signs indicative of brain tumor development. GSCs that had been treated with ispinesib showed a significant impairment of secondary tumor initiation, further confirming a compromised stem cell phenotype (FIG. 5A). In an effort to evaluate the impact of in vivo exposure to ispinesib on tumor initiation, we treated mice with established subcutaneous tumors for 3 days with 10 mg/kg ispinesib or with vehicle. The tumors were then dissociated and FACS isolated CD133-positive GSCs from each group were intracranially injected at 1,000 or 10,000 cells per mouse (n=5 mice per group). In line with in vitro exposure to ispinesib, in vivo targeting of GSCs also reduced their tumor initiation phenotype (FIG. 5B). Altogether, these data support the efficacy of Eg5 inhibition against GSCs in vivo.

### Eg5 Inhibition Reduces Tumor Growth and Improves Survival

To evaluate the efficacy of Eg5 inhibition in a more clinically relevant system, we utilized an orthotopic xenograft model to systemically deliver ispinesib. We first validated that ispinesib reaches an intracranial tumor by administering a 10 mg/kg dose to an orthotopic tumor-bearing mouse and harvesting the tumor 5 hours later. The tumor was sectioned and probed for tubulin to evaluate for the presence of mono-astral spindles, a hallmark of Eg5 inhibition. As FIG. 5C demonstrates, the tumor contained numerous cells with mono-astral spindles confirming that sufficient systemically administered ispinesib reached the tumor, inhibited its target, and produced the expected phenotype. Vehicle treated intracranial tumors did not show mono-astral spindle formation (data not shown). This finding prompted us to evaluate the effect of systemically administered ispinesib on survival in an orthotopic GBM model, in which 10,000 luciferase-expressing GSCs were injected intracranially in a group of 20 NSG mice. After waiting 7 days post injection, we confirmed the presence of tumor by demonstrating a luminescence signal (data not shown). Mice were then randomized into two groups of 10, with one group receiving vehicle (DMSO) and the other ispinesib (10 mg/kg, administered on a q4d×6 dosing schedule), both administered by intraperitoneal injection. We monitored each cohort daily for weight loss and neurological signs indicative of brain tumor development. Mice treated with ispinesib demonstrated a significant survival advantage over those treated with vehicle (p<0.001) with a median survival of 36 days versus 24 days for the DMSO vehicle cohort (FIG. 5D). These data therefore demonstrate that Eg5 inhibition is effective in a preclinical model, where it appears to prolong tumor latency and survival.

### Eg5 is Required for the Motility and Morphogenesis of GSCs and in Endothelial Cells

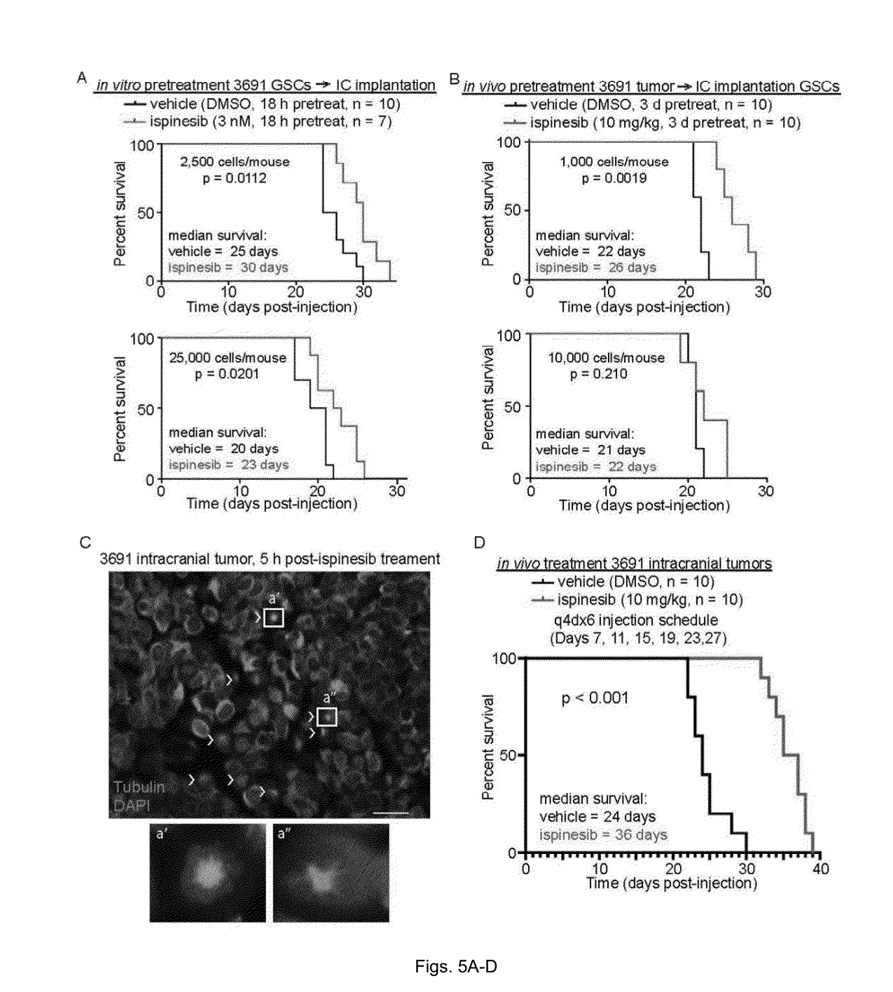

While Eg5 has been considered to be an essential component in mitosis, there have been a number of reports suggesting it has a role as well outside of the mitotic cycle, including in the regulation of cell motility, axonal branching, and angiogenesis. However, it should be noted that cell motility ceases during M phase, when both the microtubule- and actin-based cytoskeletons are recruited to form mitotically important structures, including the spindle and the cytokinetic ring. Hence, studying a putative extra-mitotic role for Eg5 in cell motility or morphogenesis requires insuring that any effects of Eg5 inhibition on these processes occur in cells that are outside of M phase. In order to accomplish this, we treated GSCs with a double-thymidine block in order to arrest them at the G1/S boundary and monitored their cell cycle state through the following experiments. Approximately twelve hours later, when the cells had fully recovered from the arrest and had begun to enter G1, they were plated in a 3 μm pore-sized Transwell migration assay in the presence of increasing concentrations of ispinesib (FIG. 6A). 8 hours later, the cells were fixed and nuclei stained with DAPI for visualization and quantification of cells that had migrated through the membrane. We found that ispinesib could completely block Transwell migration under these conditions with an EC₅₀ of 67.2 nM (FIG. 6B). In the presence of 200 nM ispinesib, approximately 6-fold fewer cells migrated per field through the Transwell compared to vehicle control (FIG. 6C). We then wanted to evaluate the impact of Eg5 inhibition on invasion in a more biologically-relevant, ex vivo setting. We therefore examined the effect of ispinesib on tumor dispersion using a rodent GBM model produced by intracranially injecting a bicistronic retrovirus encoding for PDGF and GFP in 3 day old rat pups (FIG. 6D). As we have previously shown, tumors that are histologically identical to GBM and which have a proneural GBM gene expression signature develop robustly in this rodent model within 7-10 days post injection. We generated 300 μm thick sections through the tumor-bearing portion of the brain at days 5, 6 and 7 post-injection, and monitored the effect of ispinesib (200 nM) or vehicle (DMSO) on the migration of the fluorescent tumor cells using time-lapse microscopy, as previously described (refs). Slices were counterstained with rhodamine-labeled isolectin b4 in order to fluorescently label tumor associated microglial cells. Individual green fluorescent tumor cells (n=50) from the treatment and control groups from 6 separate recordings were tracked over the course of 9.5 hours. Representative videos of treatment and control brain slices are shown in the Supplement Movies 1 and 2. Cell tracks were transposed to a common origin to generate Wind Rose plots for ispinesib and vehicle-treated samples (FIG. 6E). These demonstrate a clear reduction in tumor dispersion, which is supported by a plot of mean squared displacement (MSD) versus time (FIG. 6F). These plots of MSD(t) were fit to a Persistent Random Walk model (REFS), which relates MSD to time by the following relationship:

MSD=2S²P·[t−P(1−exp(−t/P))]

where S is cell velocity and P is the persistence time. This analysis reveals that ispinesib reduces cell velocity by approximately two-fold (21.9±0.1 μm/hr for control, versus 11.3±0.2 μm/hr for ispinesib treated) but has little effect on persistence time (1.9±0.02 hrs for control versus 2.3±0.1 hrs for ispinesib treated). Since MSD varies as the square of velocity, a two-fold reduction in the latter with ispinesib corresponds to the approximately four-fold reduction in mean squared displacement.

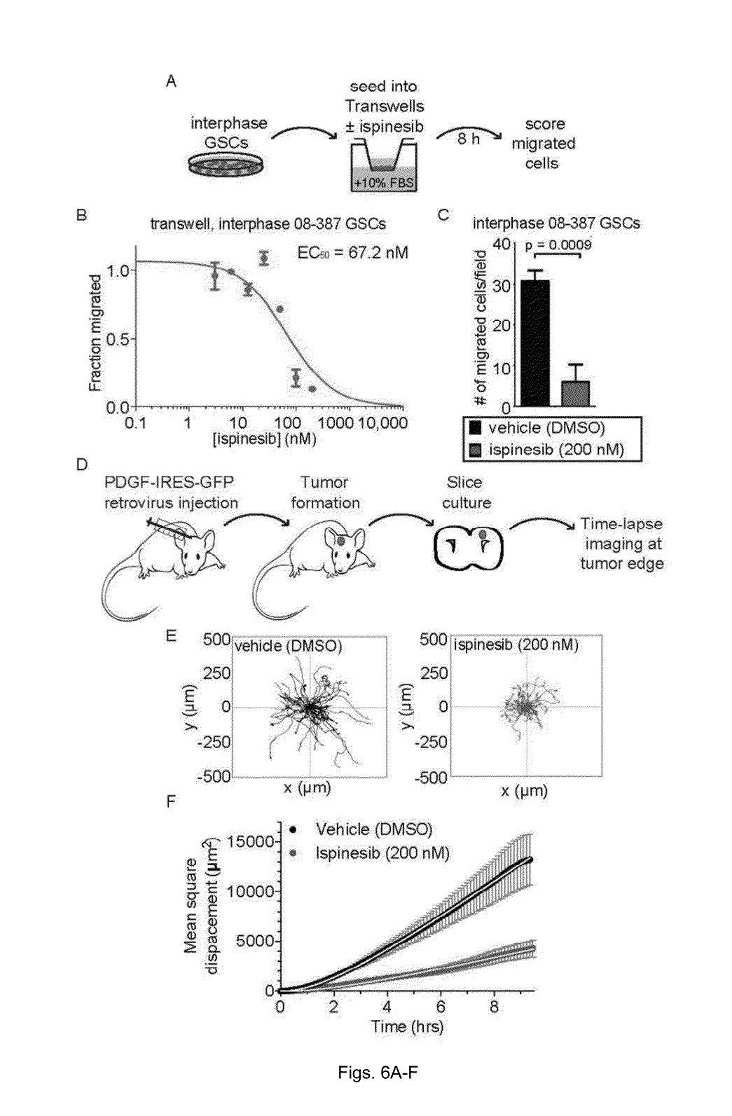

A cellular morphologic event that is common to cell motility, axonal branching, and angiogenesis is the formation of cytoplasmic projections. One process that depends critically on these projections is endothelial tube formation—generally considered to be an in vitro equivalent of the early morphologic stages that underlie angiogenesis. We examined the effect of ispinesib on tube formation by human brain microvascular endothelial cells cultured on Matrigel. These endothelial cells were labeled with CellTracker Green CMFDA, plated onto Matrigel to stimulate tube formation in the presence of vehicle (DMSO) or ispinesib (200 nM), and monitored over a 6-hour time course. We chose this time frame to minimize any effects of ispinesib on tube formation due to a G2/M arrest. Ispinesib treated cells failed to properly organize into tubes whereas distinct endothelial tubes were already formed in the vehicle treated cells at 3 hours with full tube formation seen by 6 hours. We have also found that the effect of ispinesib on cellular process formation is not limited to endothelial cells. We seeded GSCs enriched in G1 at a low density in the presence of vehicle or 200 nM ispinesib and monitored the kinetics of cytoplasmic process formation over a 6-hour time course. As FIGS. 7A and 7B illustrate, process length in GSCs was appreciably attenuated by drug treatment (p<0.0001) in a time dependent manner.

### Eg5 Regulates Plus-End Microtubule Growth

Eg5 functions in mitosis as a plus-end directed processive mitotic kinesin. Several other mitotic kinesins are known to affect the dynamics of tubulin polymerization including members of the kinesin 7, 8, 13, and 14 families. More recently, it has been reported that dimeric constructs of human Eg5 have an unusually long dwell time at microtubule plus ends and can induce tubulin protofilament assembly there, acting in essence as a tubulin polymerase. Eg5 inhibitors, such as ispinesib, may block cell motility by reducing microtubule lengthening at the leading edge of migrating cells. We therefore examined the effect of ispinesib on the content of microtubules in GSCs. We plated GSCs enriched in interphase via cell synchronization in a wound assay in order to induce cell polarization toward the cell free zone over the course of 6 hours. Cells were treated with either DMSO vehicle or 200 nM ispinesib during this time course, and then fixed, permeabilized and stained with anti-tubulin primary antibody followed by secondary detection with AlexaFluor488 that has fluorescence emission peak of around 519 nm. While vehicle treated cells at the wound edge displayed polarized arrays of microtubules, microtubules in ispinesib treated cells were less structured and few cells had developed processes indicative of a leading edge (FIG. 7C). We used spinning disc confocal microscopy to capture the AlexaFluor488 signal from the secondary antibody targeting tubulin then used post-acquisition software to measured the pixel intensity of the signal only at the primary cell layer of the wound assay leading edge. This allowed us to determine that there is a significant decrease in polymerized tubulin in cells treated with ispinesib (FIG. 7D).

Cell polarization relies on the establishment and maintenance of a dynamic microtubule array, so we next sought to determine whether the elevated interphase Eg5 that we see in GSCs (FIGS. 2A-C) directly affects microtubule assembly. We transfected GSCs with EB1-EGFP (FIG. 7E), which tracks growing microtubule tips (Seetapun et al, Curr Biol, 2012), and obtained streaming time-lapse images of EB1 ‘comets’, reflecting growing microtubule plus ends, within these cells. We measured the velocities and times of individual growth events using TipTracker (Prahl et al, Meth Enzymol, 2014) with a modification that enables tracking of EB1-EGFP signal (Seetapun et al, Curr Biol, 2012). Addition of 200 nM ispinesib reduced microtubule growth times after 7 hours of incubation, with a similar decrease observed after 30 minutes that did not reach statistical significance, compared to vehicle-treated controls (FIG. 7F), suggesting that inhibition of Eg5 activity increases catastrophe frequency in these cells. In cells with active Eg5, this modest protection against catastrophe may enable microtubules to grow into the longer processes to support cell protrusion. We also observed a significant increase in EB1 comet velocity in ispinesib-treated cells after 7 hours, suggesting that Eg5 inhibition results in a delayed cellular compensatory response. Increased growth velocity is inconsistent with Eg5 as a polymerase, although it should be noted that budding yeast isoforms of kinesin-5 are thought to act as depolymerases during mitosis (Gardner et al., Cell, 2008). Furthermore, the effects of individual kinesins on microtubule assembly (e.g., acting as a polymerase or depolymerase) may change depending on environmental conditions or modifications of tubulin polymer (Gardner, Odde, & Bloom, Trends Cell Biol, 2008). In addition to polymerization, net transport of microtubules by motor-based forces or connection to the actin cytoskeleton may also contribute to the apparent rate of microtubule growth (Bicek et al., MBoC, 2009). Kymographs of individual growth events reveal apparent motion of EB1 comets relative to polymerization in both control and ispinesib-treated GSCs (Figure S5B and Movies S3 and S4), suggesting that EB1 comet velocity is also influenced by polymer transport. Growing microtubules in dynamic cellular processes such as neuron growth cones may encounter actin retrograde flow from the cell cortex, which alters the apparent growth rate due to retrograde transport of microtubule polymer (Schaefer et al., Dev Cell, 2008; Seetapun & Odde, Curr Biol, 2010). In neurons, Eg5 also acts as a “brake” on axonal polymer transport by resisting sliding of parallel or antiparallel aligned microtubules (Falnikar et al., MBoC, 2011). We therefore suggest that the increase in growth velocity following ispinesib treatment is influenced by a loss of Eg5-mediated microtubule cross-linking that would otherwise resist bending or sliding, and that the observed reduction in growth time is a more robust measure of the direct effect of Eg5 inhibition on microtubule dynamics.

### Eg5 Inhibition Disrupts the Activity of the Tubulin Transport Protein CRMP2

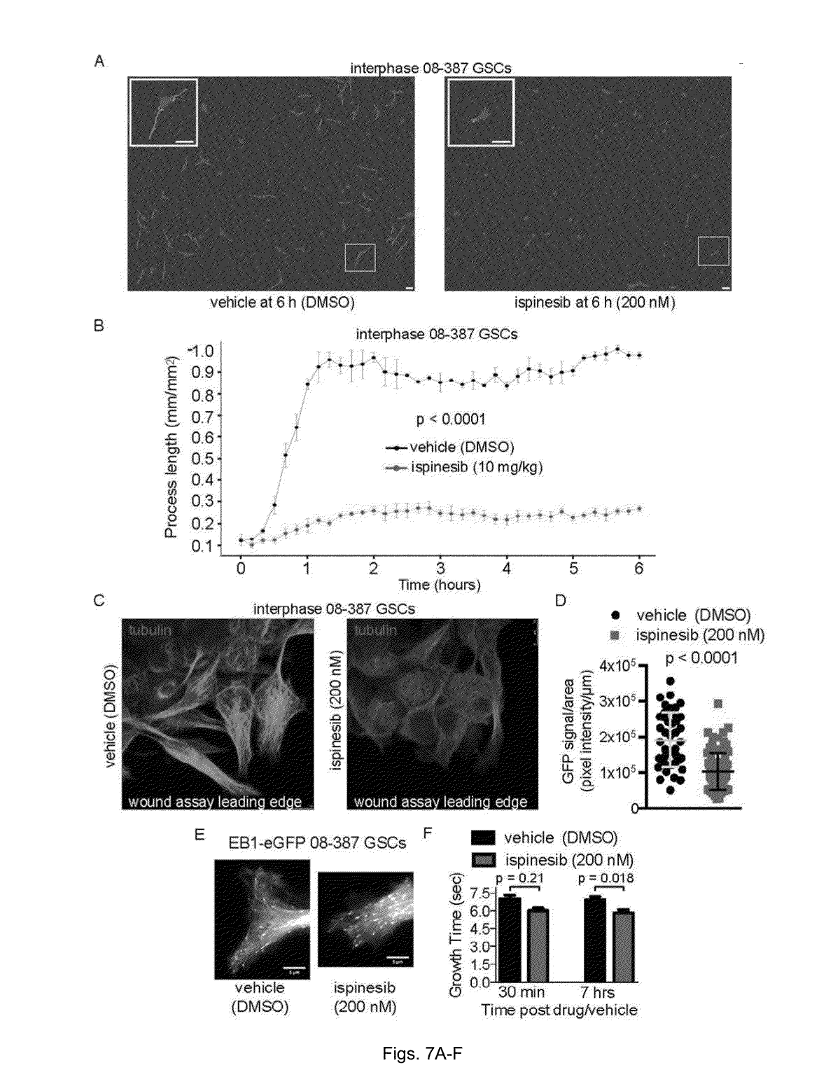

Our results suggest that the Eg5 controls cell motility and morphology by modulating tubulin polymerization dynamics. Microtubule dynamics are regulated by a wide array of cytoskeletal-associated signaling pathways, and our data does not indicate if the corresponding kinases or structural proteins work in conjunction with Eg5 to modulate this process. We therefore generated lysates of interphase GSCs treated with vehicle or 200 nM ispinesib for 7 hours and probed them with a commercially available, cytoskeletal phospho-antibody array. This revealed that ispinesib produces no significant changes in phosphorylation for 141 proteins evaluated by this assay. However, this array does not include members of the collapsing response mediator protein (CRMP) family, which play central roles in cytoplasmic and axonal process outgrowth, precisely the functions that appear to be affected by Eg5 inhibition. One of these, CRMP2, binds to tubulin and this tubulin-CRMP2 complex is transported by kinesin 1 to the plus end of microtubules, where it in effect acts as a tubulin polymerization co-factor by supplying αβ tubulin heterodimer to the growing microtubule plus end. CRMP2 can be phosphorylated by several signaling kinases, including CDK5 and GSK3β, and phosphorylation inactivates it. In view of the connection between CRMP2, Eg5, and the microtubule plus end, we examined the effect of ispinesib on the phosphorylation status of CRMP2 in both interphase GSCs (FIG. 8A) and in human brain microvascular endothelial cells (FIG. 8B) over a 6-hour time course. As we have already shown, this is the period of time needed to see maximal effects of ispinesib on GSC and endothelial process formation. We found that treatment with ispinesib produced a significant increase in phospho-CRMP2 (T514) that was maximal between 2 and 4 hours post exposure in the GSCs. As noted in FIG. 7, this is precisely the time course over which the effect of ispinesib on cell process formation in endothelial cells and GSCs is seen.

These results suggest a mechanism, in which CRMP2 delivers tubulin dimers to the plus ends of microtubules, increasing local concentrations of tubulin at the site of maximal microtubule dynamic instability. We suggest that Eg5, as a plus end tip tracking motor, assists in capturing free tubulin and incorporating it into growing protofilaments, consistent with recent reports that Eg5 induces protofilament formation at the plus end of dynamic microtubules. As a corollary to this mechanism, we would also predict that interventions that impair microtubule growth, such as but not limited to inhibition of Eg5, would lead to release of CRMP2 into the cytoplasm, where CDK5 and GSK3β could phosphorylate and inactivate it. A prediction of this model is that any intervention that impacts microtubule dynamics will lead to an increase in CRMP2 phosphorylation. We tested this by treating interphase GSCs with nocodazole, in order to depolymerize microtubuless, or paclitaxel, to polymerize and stabilize microtubules, and measured the effects of these interventions on CRMP2 phosphorylation. Results indicate that both treatments induce CRMP2 phosphorylation at T514.

### Discussion

Effective treatments for malignant glioma remain challenging due to the ability of GBM cells to invade into surrounding tissue and rampantly proliferate. These two cellular processes of migration versus proliferation are often thought to be dichotomous cellular behaviors. However, the extent to which invasion and proliferation are mutually exclusive is questionable with data supporting a transient halt to allow for cell division followed by resumed migration. This observation brings to light a core mediator of cell motility and cell division, microtubules, which when committed to formation of the bipolar spindle in mitosis are precluded from aiding in migration, therefore initiating the reported transient halt in cell movement. Drugs such as vinca alkaloids and taxanes that directly target microtubules have shown activity toward many solid and hematologic cancers, but have failed to demonstrate efficacy for GBM. This may be in part due to inefficient entry into the central nervous system but also the greater issue of neurotoxicity toward the heavily microtubule dependent axons within neurons. The exploration of more recently developed drugs that instead target microtubule associated proteins is extremely limited in GBM. Our efforts demonstrate that targeting the kinesin Eg5 offers a dual therapeutic approach to impede both proliferation and invasion in GBM. These finding yield substantial clinical implications as currently there are no drugs able to halt invasion and create a tumor mass that could be more easily surgically resected and none of the current cytotoxic drugs aimed at proliferation can target invading cells. Therefore, small molecule inhibition of Eg5 may constitute a new class of directed therapeutics that is both anti-proliferative and anti-invasive.

We validate that Eg5 is highly elevated in GBM over normal brain Peter—some discussion about what the bioinformatics on Eg5 means for GBM.

Furthermore, Eg5 demonstrates intratumoral heterogeneity with higher expression in the GSC subpopulation. Although some level of differential transcription between GCS s and NSTCs may be a contributing factor, our data clearly indicate persistent Eg5 protein levels in the GSCs attributable to attenuated protein turnover. The APC/C is a known tumor suppressor yet we understand little regarding its role in GSC or GBM biology, calling for further studies. What our data does indicate, however, is that the therapeutic resistance reported for GSCs does not hold for small molecule inhibition to Eg5. Viability, self-renewal and tumor initiation are all compromised for GSCs following Eg5 inhibition. Importantly, our studies validate that GSCs that had seen drug in vivo are compromised in tumor initiation, a possible surrogate for reduced contribution to tumor recurrence. We also validated an impact on survival in a preclinical model that is based on a GSC-derived tumor. Related to this is that therapeutic efficacy is not restricted to GSCs. With an expanded appreciation of the cellular plasticity for NSTCs under certain microenvironmental conditions, such as hypoxia and acidic stress, a drug that only targets one cellular population could fail in the clinic. Our data highlight a broad utility of Eg5 inhibition with compromised viability of both GSCs and NSTCs.

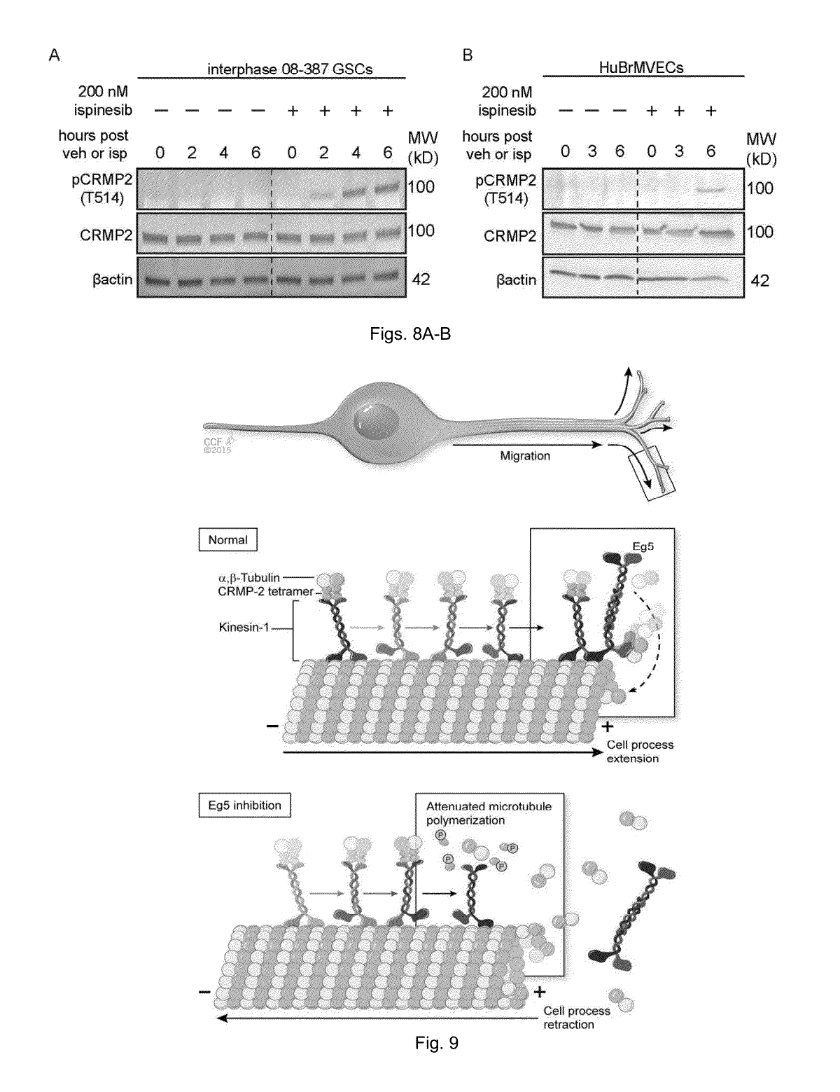

By utilizing our cell cycle enrichment paradigm, our studies allow for delineation of the role of Eg5 in mitosis from Eg5 in invasion. We have elucidated a role for Eg5 in building and maintain a leading process in motile GSCs. We show disruption of the CRMP2 tubulin deposition axis along with direct impediment of microtubule polymerization. We therefore propose a model (FIG. 9)

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating glioblastoma multiforme (GBM) in a subject in need thereof comprising, administering to the subject a therapeutically effective amount of an Eg5 inhibitor.

2. The method of claim 1, wherein the Eg5 inhibitor inhibits GBM metastasis in the subject.

3. The method of claim 1, the glioblastoma in the subject characterized by the presence of glioma cancer stem cells.

4. The method of claim 1, wherein the GBM comprises GBM of the brain.

5. The method of claim 1, wherein the subject is a human.

6. The method of claim 1, further comprising surgical resection of a subject's GBM tumor.

7. The method of claim 1, the Eg5 inhibitor comprising a small molecule.

8. The method of claim 7, the small molecule targeting a motif in the catalytic domain of Eg5.

9. The method of claim 7, wherein the Eg5 inhibitor is selected from the group consisting of:

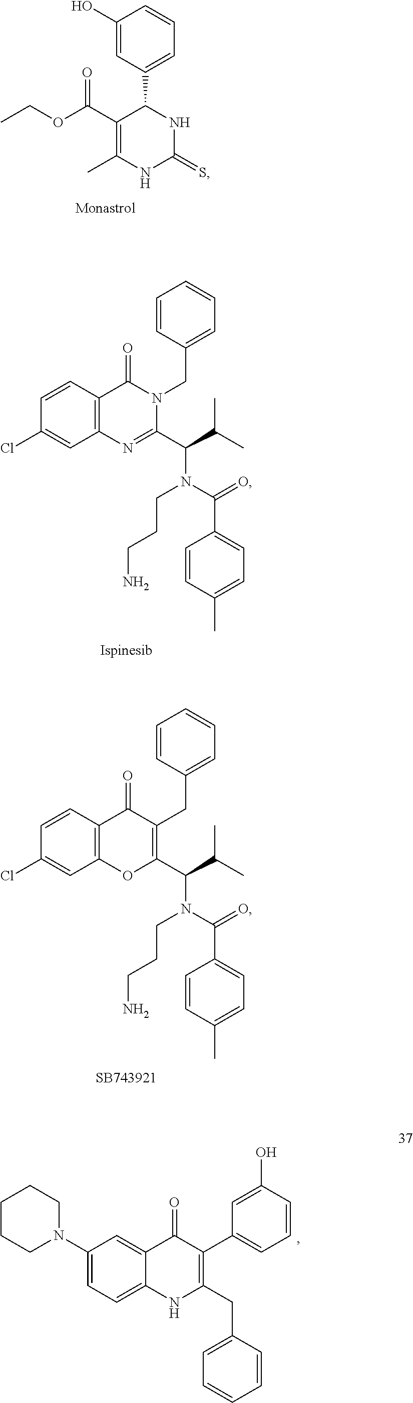 [SMILES: CCOC(=O)C1=C(C)NC(=S)N[C@H]1c1cccc(O)c1 ; Cc1ccc(C(=O)N(CCCN)[C@@H](c2nc3cc(Cl)ccc3c(=O)n2Cc2ccccc2)C(C)C)cc1 ; Cc1ccc(C(=O)N(CCCN)[C@@H](c2oc3cc(Cl)ccc3c(=O)c2Cc2ccccc2)C(C)C)cc1 ; O=c1c(-c2cccc(O)c2)c(Cc2ccccc2)[nH]c2ccc(N3CCCCC3)cc12]

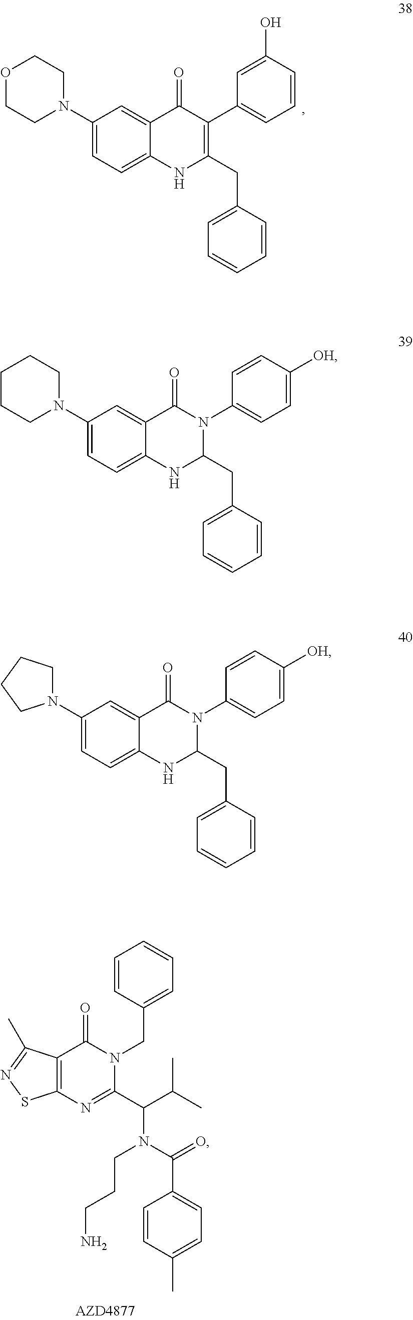 [SMILES: O=c1c(-c2cccc(O)c2)c(Cc2ccccc2)[nH]c2ccc(N3CCOCC3)cc12 ; O=C1c2cc(N3CCCCC3)ccc2NC(Cc2ccccc2)N1c1ccc(O)cc1 ; O=C1c2cc(N3CCCC3)ccc2NC(Cc2ccccc2)N1c1ccc(O)cc1 ; Cc1ccc(C(=O)N(CCCN)C(c2nc3snc(C)c3c(=O)n2Cc2ccccc2)C(C)C)cc1]

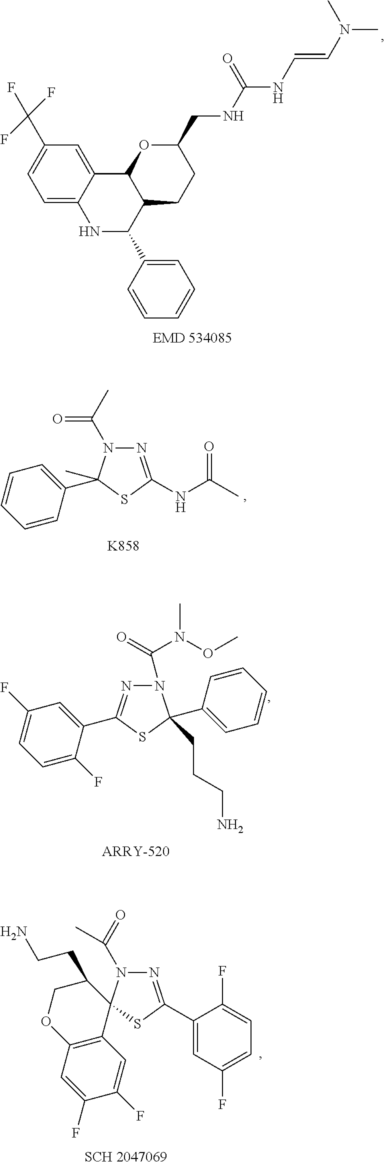

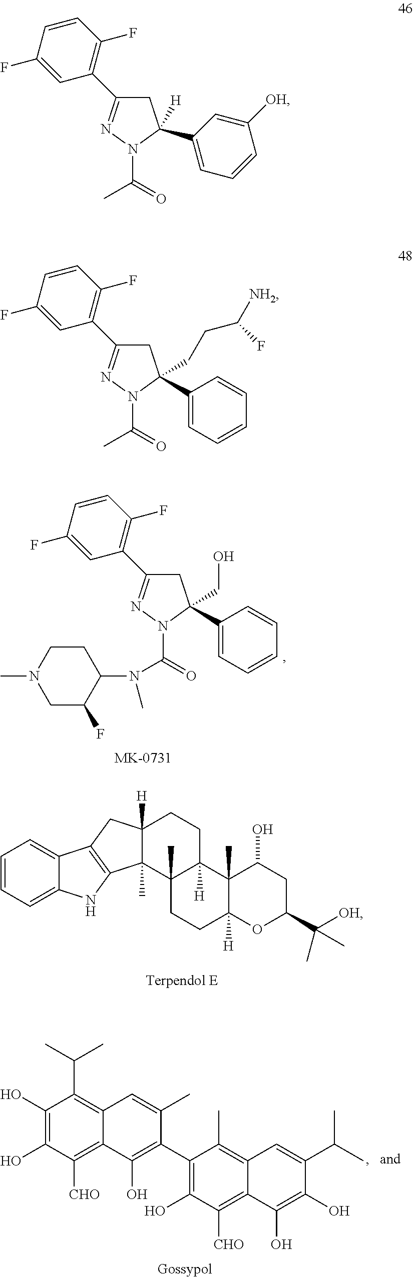

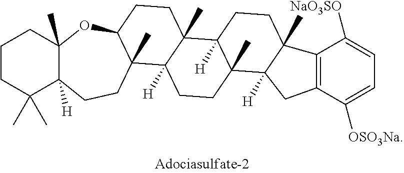

10. The method of claim 4, the Eg5 inhibitor comprising ispinesib.

11. The method of claim 1, wherein the Eg5 inhibitor is administered systemically.

12. The method of claim 1, wherein treating GBM in a subject in need thereof comprises preventing cancer metastasis in the subject, wherein the subject has been diagnosed with GBM.
